(19) United States
(12) Patent Application Publication
(10) Pub. No.: US 2012/0178116 A1
(43) Pub. Date: Jul. 12, 2012

(54) POLYPEPTIDES FOR IDENTIFYING FUNGICIDALLY ACTIVE COMPOUNDS

(76) Inventors: Christoph Andreas Braun, Dusseldorf (DE); Peter Schreier, Koln (DE); Klaus-Gunther Tietjen, Langenfeld (DE); Michael Edmund Beck, Monheim (DE); Amos Mattes, Langenfeld (DE); Jorg Nico Greul, Leichlingen (DE); Oliver Gutbrod, Langenfeld (DE); Sandra Tuckmantel, Leverkusen (DE); Daniel Raun, Worms (DE)

(21) Appl. No.: 13/330,873

(22) Filed: Dec. 20, 2011

Related U.S. Application Data
(60) Provisional application No. 61/429,784, filed on Jan. 5, 2011.

(51) Int. Cl. C12Q 1/48; C12N 15/63; C12N 9/12; C12N 1/19; C12N 1/21; C12N 1/15; C12N 5/10; C12N 15/54; C07K 16/40

(52) U.S. Cl. 435/15; 536/23.2; 435/320.1; 435/194; 530/389.8; 435/252.3; 435/254.11; 435/419; 435/325; 435/367; 435/369; 435/357; 435/354; 435/365; 435/349; 435/352; 435/358; 435/252.33; 435/252.35; 435/254.2; 435/348

(57) ABSTRACT

The invention relates to a combination of polypeptides from phytopathogenic fungi having the biological activity of an aurora kinase and the function of an aurora kinase activator, to nucleic acids coding therefore, to the use of the polypeptides and nucleic acids for identifying modulators of an aurora kinase, to processes for identifying such modulators and to the use of these modulators as fungicides.

The present application relates inter alia to a process for identifying fungicides, comprising (a) bringing a combination of a polypeptide having the biological activity of an aurora kinase (AK) and a polypeptide having the biological function of an aurora kinase activator (AKA) into contact with a chemical compound or a mixture of chemical compounds, (b) comparing the biological activity of the AK from the combination of (a) in the presence of the chemical compound or the mixture of chemical compounds to the biological activity of the AK from the combination of (a) in the absence of the chemical compound or the mixture of compounds, and, optionally, (c) determining the chemical compound from a mixture which inhibits the biological activity of the AK; where a polypeptide having the biological activity of an AK comprises (i) an amino acid sequence SEQ ID No: 2; or (ii) an amino acid sequence having at least 80%, at least 85%, at least 90%, at least 95% and in particular at least 98% identity to the amino acid sequence SEQ ID No: 2; and where a polypeptide having the biological function of an AKA comprises (I) an amino acid sequence SEQ ID No: 6; or (II) an amino acid sequence having at least 70%, at least 75%, at least 80%, at least 85%, at least 90%, at least 95% and in particular at least 98% identity to the amino acid sequence SEQ ID No: 6. In addition, the present application discloses the use of a combination of a polypeptide having the biological activity of an AK and a polypeptide having the biological function of an AKA as described above for identifying fungicidal compounds, nucleic acids comprising a sequence coding for an AKA as described above; a polypeptide encoded thereby, a combination comprising the polypeptides described above having the biological function of an AKA and the biological activity of an AK and also a process for preparing this combination.

The present description quotes a number of documents including patent applications and instructions for use. Although the disclosure of these documents is considered to be irrelevant for the patentability of the present invention, it is nevertheless incorporated into the present application in its entirety by reference. In particular, all cited documents arc incorporated into the present application to the same extent as if each document were described specifically and individually as incorporated herein.

In principle, the basics of how to identify fungicides via inhibition of a defined target by these fungicides are known, for example, from WO 2005/042734, WO 03/054221, WO 00/77185 and U.S. Pat. No. 5,187,071. In general, there is a great need to find enzymes involved in essential metabolic pathways. These enzymes may represent novel target proteins or targets for fungicides allowing the control of, for example, resistances, and generally the production of fungicides having improved ecological and toxicological safety, at lower expense. Frequently, it is a problem that the effects of inhibiting an enzyme in a metabolic pathway can be circumvented by using alternative metabolic pathways.

In practice, the detection of novel targets is associated with great difficulties, since the inhibition of an enzyme which is a component of a metabolic pathway frequently has no further effect on growth or infectiousness of the pathogenic fungus. This may be due to the fact that the pathogenic fungus side-steps to alternative unknown metabolic pathways or that the enzyme inhibited is not limiting for the metabolic pathway. Accordingly, the suitability of a gene product as a target cannot be predicted even if the gene function is known.

N-substituted diaminopyrimidines are a class of low-molecular-weight compounds which have been described as inhibitors of human kinases (Argiriadi et al., 2010, Bioorg. Med. Chem. Lett., 20(1), 330-333). In addition, it has been found that they have fungicidal action (WO 2008/107096).

Whereas in humans the focus is on the inhibition of deregulated kinase activity for curing diseases, fungi such as, for example, Neurospora crassa can be controlled by inhibiting their normal kinase activity (Pillonel, 2005, Pest Manag. Sci., 61, 1069-1076).

Aurora kinases play an important role in cell growth. In the human genome, there are three different aurora kinases: A, B and C. All three aurora kinases are involved in particular in cell division (cytokinesis) and segregation of the chromosomes. In other organisms, too, aurora kinases. play an essential role in these processes. A loss of their function has serious consequences caused by an uneven distribution of the chromosomes (Bischoff and Plowman, 1999, Trends Cell Biol., 9, 454-459). Whereas three different isoforms of aurora kinase are present in Metazoa (A, B and C), in yeasts such as S. cerevisiae or Schizosaccharomyces pombe there is only one aurora kinase (Honda et al., 2003, Mol. Biol. Cell, 14, 3325-3341).

There is a constant need to identify novel targets and fungicides directed against these targets, for example to prevent resistances.

This object is achieved by the present claims of the invention.

The present application completely describes, for the first time, an associated pair of a polypeptide having the biological activity of an AK and a polypeptide having the biological function of an AKA of a phytopathogenic fungus, in the present case U. maydis, where in the absence of the AKA the AK has no measurable kinase activity.

Homology comparison of the sequence SEQ ID No: 1 from U. maydis showed great similarity with known sequences coding for human AK or with sequences coding for AK from other fungi. Here, a sequence from Puccinia graminis showed greatest homology to the sequence SEQ ID No: 1 from U. maydis (see Table 1).

TABLE 1

| | |
|---|---|
| P. graminis | homolog: aurora protein kinase, E value 3e−114, |
| | 65% identity, 79% similar amino acids |
| S. pombe | homolog: aurora B/Ark1, E value 6e−99, 59% identity, |
| | 77% similar amino acids |
| man | homolog: aurora A, E value 3e−89, 60% identity, |
| | 75% similar amino acids |

All known AKs of type A and B show aurora kinase activity even without addition of an AKA; however, an AKA may increase the activity of a corresponding AK by a factor of from 7 to 15 (Honda et al., 2003, Mol. Biol. Cell, 14, 3325-3341; Kang et al., 2001, J. Cell Biol., 155, 763-774).

However, in contrast to all AKs known to date, the translation product of SEQ ID No: 1 (SEQ ID No: 2) shows no kinase activity. Even in the presence of the specific known AKA (INCENP sequence) from S. pombe (Leverson et al., Mol. Biol. Cell., 2002, 13(4), 1132-43), the amino acid sequence of which coding for an AK has high similarity to SEQ ID No: 1, it was not possible to measure an aurora kinase activity of a protein having the SEQ ID No: 2.

However, surprisingly, it has been found that a protein having SEQ ID No: 2 has kinase activity in the presence of a protein having SEQ ID No: 6 or a protein comprising an amino acid sequence SEQ ID No: 6, such as, for example, SEQ ID No: 4, SEQ ID No: 14, or SEQ ID No: 16.

At the MIPS (Munich Information Centre for Protein Sequences (© 2003 GSF-Forschungszentrum für Umwelt and Gesundheit, GmbH Ingolstädter Landstraβe 1, D-85764 Neuherberg, Germany)), SEQ ID No: 6 is listed only as a potential protein.

### SEQUENCES AND FIGURES

SEQ ID No: 1 nucleic acid sequence AK (U. maydis) without TAG

SEQ ID No: 2 amino acid sequence AK (U. maydis) without TAG

SEQ ID No: 3 nucleic acid sequence AKA (U. maydis) without TAG

SEQ ID No: 4 amino acid sequence AKA (U. maydis) without TAG

SEQ ID No: 5 nucleic acid sequence AKA (C terminus (U. maydis)) without TAG

SEQ ID No: 6 amino acid sequence AKA (C terminus (U. maydis)) without TAG

SEQ ID No: 7 nucleic acid sequence primer AK forward

SEQ ID No: 8 nucleic acid sequence primer AKA forward

SEQ ID No: 9 nucleic acid sequence primer AK reverse

SEQ ID No: 10 nucleic acid sequence primer AKA reverse

SEQ ID No: 11 nucleic acid sequence AK (U. maydis) with TAG

SEQ ID No: 12 amino acid sequence AK (U. maydis) with TAG

SEQ ID No: 13 nucleic acid sequence AKA (U. maydis) with TAG

SEQ ID No: 14 amino acid sequence AKA (U. maydis) with TAG

SEQ ID No: 15 nucleic acid sequence AKA (C terminus (U. maydis)) with TAG

SEQ ID No: 16 amino acid sequence AKA (C terminus (U. maydis)) with TAG

FIG. 1: Radioactive activity test of (A) purified SEQ ID No: 12, (B) purified SEQ ID No: 16, (C) a combination of separately purified SEQ ID No: 12 and SEQ ID No: 16, (D) a combination of co-purified SEQ ID No: 12 and SEQ ID No: 16, (E) a combination of co-purified SEQ ID No: 2 and SEQ ID No: 6 (co-purified SEQ ID No: 12 and SEQ ID No: 16 with subsequent Tag removal)

FIG. 2: Radioactive activity test of (A) a combination of co-purified SEQ ID No: 12 and SEQ ID No: 16, (B) SEQ ID No: 12, (C) SEQ ID No: 16, (D) negative control (Mastermix), (E) a combination of separately purified SEQ ID No: 12 and SEQ ID No: 16, (F) a combination of SEQ ID No: 12 and Pic1 from S. pombe, (G) a combination of SEQ ID No: 12 and human GST-INCENP (Cat #12-534, Millipore)

FIG. 3: Sequence comparison of Pic1 from S. pombe and um03367 (SEQ ID NO: 6=C terminus of SEQ ID NO: 4) showed that on a small part consisting of amino acids 925-1018 of INCENP from S. pombe and amino acids 1424-1575 of SEQ ID NO: 6 there was 39% identity (based on amino acid positions 925-1019 from S. pombe).

FIG. 4: Kinetics of the phosphorylation of substrates for human aurora kinase A in IMAP by a combination of co-purified SEQ ID No: 12 and SEQ ID No: 16. SEQ ID No: 16 alone shows no activity. Human aurora kinase A+ human INCENP were used as positive control.

FIG. 5: Growth inhibition (ED₅₀ determination) of U. maydis with Euparen and compound 1

The person skilled in the art is aware that the term “a”, as used in the present application, may mean “one (1)”, “one (1) or more” or “at least one (1)”, depending on the situation. In the context of the aurora kinase disclosed herein and the aurora kinase activator according to the invention, “a” does not refer to the number of molecules but to the number of molecular species.

It is clear to the person skilled in the art that examples given in the present application are not to be considered as limiting but rather only describe some embodiments in more detail.

The person skilled in the art is aware that all embodiments may be present on their own or in combination.

Not included are combinations which are in violation of natural laws and which the person skilled in the art would therefore have excluded based on his expert knowledge.

The term “comprising” or “comprise”, based on the present application, specifies the presence of the features, integers, steps or components listed, but does not exclude the presence of one or more additional features. Accordingly, a nucleic acid or amino acid sequence may comprise more nucleotides or amino acids than mentioned, i.e. may be part of a longer nucleic acid or amino acid sequence. An expression cassette or a chimeric gene, as described elsewhere, which is defined by its function or structure may comprise further nucleic acid sequences etc. Here, for example, the nucleic acid sequence may comprise additional nucleic acid sequences at the 3′ or at the 5′ terminus, where the length of the additional nucleic acid sequences does not exceed 6000 bp, for example 3500 bp, 1500 bp, 1000 bp, 500 by or 250 bp, at the 5′ and/or 3′ terminus. Such additional nucleic acid sequences are, for example, nucleic acid sequences coding for a tag or nucleic acid sequences present in organisms. With respect to an amino acid sequence, this may comprise additional amino acid sequences at the C and/or N terminus, the length of the additional amino acid sequences not exceeding 2000, for example 1000, 500, 250 or 100 amino acids. Accordingly, such additional amino acid sequences are, for example, a tag or an amino acid sequence present in organisms. In connection with the present application, the term “comprising” or “comprise” includes the term “consisting of” or “consist of”.

The term “fungicide” or “fungicidally” active compound, as used herein, relates to chemical compounds suitable for controlling fungi, in particular those which infest and damage plants, plant parts or plant products or reduce their yield or value. The plant parts mentioned include, for example, leaves, seeds and crops (such as, for example, berries, fruit, cereal grains). The plant products mentioned include raw materials or substances obtained from the plants such as, for example, woods or fibres.

The term “bringing into contact” includes a contact between the combination disclosed herein and one or more chemical compounds to be tested in any form under all conditions which ensure an activity of the AK according to the invention present in the combination. For in vitro tests using a combination in liquid form (in other words a solution comprising the combination according to the invention), the chemical compound(s) to be tested is/are added to the combination according to the invention or vice versa. For example, the chemical compound(s) to be tested is/are added in the form of a solution to a solution comprising the combination according to the invention. For tests with the combination where both the AK according to the invention and the AKA according to the invention are expressed in a host cell, the chemical compound may be added to the host cell.

A combination is any possible mixture comprising an AK according to the invention and an AKA according to the invention. The combination may be liquid, solid or a combination thereof. In other words, a combination according to the invention may, for example, be a solution, consist of solids, be an emulsion or dispersion or an aerosol. In the context of the process according to the invention, the combination according to the invention is usually present in liquid form. For storage, it may be either in liquid or else in solid form. A combination in liquid form may comprise, for example, only water and/or an organic solvent, but in addition also one or more salts in varying concentrations and/or a buffer substance and further auxiliaries such as solubilizers or stabilizers.

Suitable salts are sufficiently known to the person skilled in the art and include, for example, Na salts such as NaCl, or divalent metal salts such as Mg²⁺ or Mn²⁺ salts (for example MgCl₂ or MnCl₂).

Suitable buffers are, for example, tris(hydroxymethyl)aminomethane (IRIS), 4-(2-hydroxyethyl)-1-piperazineethanesulphonic acid (HEPES), 4-(2-hydroxyethyl)piperazine-1-propanesulphonic acid (HEPPS), 3(N-morpholino)propanesulphonic acid MOPS or 2(N-morpholino)ethanesulphonic acid (MES).

In the context of the present application, the term “polypeptide” (exchangeable with the term “protein”) describes a group of molecules consisting of more than 30 amino acids, whereas the term “peptide” describes molecules consisting of up to 30 amino acids. Polypeptides and peptides may form dimers, trimmers or higher oligomers, i.e. the resulting structures consist of more than one polypeptide/peptide molecule. The polypeptides or peptides forming such dimers, trimmers, etc., may be identical or nonidentical. The corresponding structures of a higher order are therefore referred to as homo- or heterodimers, homo- or heterotrimers, etc. The terms “polypeptide” and “peptide” also apply to polypeptides or peptides modified in a natural manner, for example by glycosylation, acetylation, phosphorylation, etc. Such modifications are sufficiently known.

The polypeptides according to the invention may be present in the form of “mature” proteins, for example provided with the modifications described above, or as part of relatively large proteins, for example as fusion proteins. Fusion proteins are in particular AK or AKA polypeptide sequences attached to a tag, such as, for example, a His tag, GST tag (glutathione S-transferase) or MBP tag (maltose binding protein). Furthermore, they may have secretion or “leader” sequences, Pro sequences, sequences which allow simple purification, such as a plurality of histidine radicals or an MBP tag, or additional stabilizing amino acids. The proteins according to the invention may likewise be present as they are naturally present in their organism of origin, from which they may be obtained directly, for example.

“Tag” refers to a peptide or polypeptide whose coding nucleic acid sequence may be fused to the nucleic acid sequence of a polypeptide having the activity of an AK or the function of an AKA, directly or via a linker, using customary cloning techniques. A tag may serve for isolation, enrichment and/or targeted purification of a recombinant (target) protein by affinity chromatography, for example whole cell extractions, and/or to better solubilize recombinant (target) proteins. The linker mentioned above may advantageously comprise a protease cleavage site (for example for thrombin or factor Xa), whereby the tag may, when required, be cleaved from the target protein. Examples of customary tags are “His tag”, for example from Qiagen, “GST tag”, for example from Clontech, “MBP tag”, for example from Promega, “Strep tag”, for example from IBA Biotagnologies, “Myc tag” or “CBD tag”. A tag can be attached at the 5′ or 3′ end of a coding nucleic acid sequence having the sequence coding for the target protein. In one embodiment of the invention, an AK and/or an AKA is a recombinant protein (fusion protein) having an MBP tag.

An AK catalyses the phosphorylation of a specific protein substrate such as, for example, basic myelin protein, on serine and/or threonine residues, ATP acting as a donor substrate (also referred to as “AK activity”). The enzymatic activity of an AK such as, for example, the AK described in the present application can be determined in an activity test via the increase of product, the (significant) decrease of substrate (or starting material) or the decrease of a specific cofactor or via a combination of at least two of the parameters mentioned above as a function of a defined time span, or reaction time. It is known to the person skilled in the art that some proteins require essential ions such as divalent metal ions (for example Mg²⁺ or Mn²⁺) for their activity/function. In such a prior to activity measurements, the person skilled in the an will obviously add these ions, for example in the form of their salts (such as MgCl₂) and in an appropriate concentration (for example 5 mM, 10 mM, 20 mM, 50 mM), to a solution comprising the corresponding protein, for example an AK or AKA according to the invention. Preferably, such essential ions are present even during purification and/or during the production of the proteins in the media in question. Accordingly, in the present application all solutions comprise at least one divalent metal ion species such as, for example, Mg²⁺.

Here, the term “reaction time” refers to the time required for carrying out an activity test until a significant statement with regard to an activity is obtained, and it depends both on the specific activity of the protein employed in the test and on the method used and the sensitivity of the instruments used. The person skilled in the art is familiar with how to determine reaction times. In the case of processes based on photometric methods for identifying substances having fungicidal activity, the reaction times are generally from >0 to 600 minutes.

A “significant decrease” with regard to the activity of an AK is an activity decrease of the AK, mixed with a test compound, and AKA compared to the activity of the AK not incubated with the test compound, which is greater than any measurement error. For example, a significant decrease may be a decrease by at least 10%, at least 20%, at least 30%, at least 40%, at least 50%, at least 70%, at least 90% or even by at least 95%, at least 98% or at least 99%.

Possible assays for determining an AK activity are, for example, the radioactive determination according to Muller, P., et al. (2003, Eukaryot Cell, 2(6), 1187-99), where a subsequent quantification may be carried out, for example, using an AIDA image analyser, commercially available HTRF® Kinease Assays™ for serine/threonine kinases (Cat #62ST0PEB. Cisbio Bioassays) or commercially available IMAP assays (Molecular Devices).

An aurora kinase activator (AKA) is a polypeptide which, as biological function, may activate a polypeptide having that biological activity of an AK, for example an AK according to the invention. It is obvious that an AKA in combination with an AK according to the invention has to be able to activate this AK according to the invention. For AKs according to the invention having a low activity in the absence of an AKA according to the invention, for example, the activity of the AK according to the invention may, at a ratio of AK according to the invention to AKA according to the invention of 1:1, be improved by the AKA according to the invention compared to the activity of the same AK according to the invention in the absence of the AKA according to the invention at least by a factor of 2, 3, 4, 5, 10 or 15. AKs according to the invention without aurora kinase activity in the absence of a corresponding AKA, which can be demonstrated by a missing radioactive product band on a PAGE gel of a radioactive assay, can be activated by the presence of an AKA according to the invention, i.e. they generate a radioactively marked product band on a PAGE gel (polyacrylamide gel electrophoresis) of the same radioactive assay. For example, basic myelin protein (about 19 kDa) may be radioactively phosphorylated by an AK according to the invention in the presence of an AKA according to the invention, such that a corresponding PAGE gel with at most 500 ng of basic myelin protein (substrate of the radioactive assay) per slot has a radioactive band at about 19 kDa. A radioactive band can easily be detected by the black discolouration of a photographic film after, for example, 50 min at the corresponding site. For comparison, an identical amount of basic myelin protein not subjected to phosphorylation may be applied at the same time to a further slot of the PAGE gel in question so that possible discolourations of the film in the region of a 19 kDa band by relatively large amounts of the basic myelin protein may be excluded. An AK according to the invention activated by an AKA according to the invention may, for example, have a kinase activity different from 0, i.e. it is possible to measure a specific activity which is outside the measurement error range of an appropriate method, such as, for example, at least 0.02 U/mg, 0.2 U/mg, at least 0.5 U/mg, at least 1 U/mg, at least 5 U/mg, or even at least 50 U/mg, U/mg being defined as the amount of enzyme which, under standard conditions, converts one μmol of substrate per min.

The term “inhibit”, as used herein, refers to the direct or indirect inhibition of the enzymatic AK activity of an AK, for example an AK activated by an AKA, using a chemical compound. Such an inhibition may be specific, i.e. the inhibition of aurora kinase activity takes place at a concentration of a chemical compound (an inhibitor) which is lower than the concentration of an inhibitor required to inhibit the activity of another polypeptide unrelated to the AK. The inhibitor concentration required to inhibit the AK can, for example, be at least two times lower, at least five times lower or at least ten times lower or at least 20 times lower than the concentration of an inhibitor required to evoke an unspecific effect. An inhibition of an AK according to the invention means that the specific activity of this AK at otherwise identical conditions such as pH, substrate, amount of substrate, amount of enzyme, etc., in the presence of an inhibitor is at least 10%, 20%, 30%, 50%, 80%, 90%, 95% lower than in the absence of the inhibitor. To the person skilled in the art, it is clear that an inhibitory substance should be present in concentrations in the millimolar, μmolar or nmolar range, for example.

Chemical compounds include all molecules that can be chemically prepared or that occur naturally, for example small organochemical molecules, peptides or antibodies, bind to the AK described herein and modulate its activity. Also included are small organochemical molecules, peptides or antibodies binding to a molecule which for its part binds to the AK described herein and thereby modulates its biological activity. Chemical compounds may be natural substrates and ligands or structural or functional mimetics thereof. Chemical compounds include small molecules having a molecular weight of up to 250 Da, up to 500 Da, up to 800 Da or up to 1000 Da. Exemplary chemical compounds are N-substituted diaminopyrimidines as described in WO 2008/107096, pyridylazinylamino derivatives as described in WO 2010/055114, heterocyclyltriazinylamino derivatives as described in WO 2010/055078, or other potentially fungicidal chemical compound classes.

Mixtures of chemical compounds are mixtures of at least two different chemical compounds. In general, a mixture may be present in solid form, for example at least two mixed powders, liquid form, for example solution or emulsion, or a mixture of both, for example suspension, of at least two chemical compounds. If a mixture is present as a solution, it is, for the process according to the invention for identifying fungicides, preferably dissolved in the same solvent/buffer system, such as a solution, emulsion or suspension of the AK according to the invention and the AKA according to the invention.

Comparison of the biological activity of the AK according to the invention in a process according to the invention for identifying fungicides can be carried out, for example, by direct comparison of data obtained by one of the methods as in paragraph [0032]. This may take place, for example, by the presence of a radioactive band in a gel, this radioactive band representing a radioactively labelled product of an AK-catalysed reaction (for example basic myelin protein), or by evaluation of quantitative data obtained using an AIDA image analyser.

Determination of a chemical compound in a mixture may take place, for example, by affinity chromatography and subsequent elution and MS/MS analysis, the AK according to the invention serving as stationary phase. The person skilled in the art is familiar with methods for immobilizing proteins and conducting MS/MS analyses. (Knoth et al., Angw. Chem. 2009 48, 7240-7245, Supporting Information © Wiley-VCH 2009).

The amino acid sequence of the AK according to the invention described herein has at least 70%, at least 80%, at least 90%, at least 95% or at least 98% sequence identity to SEQ ID No: 2 or to an amino acid sequence comprising a SEQ ID No: 2, such as, for example, a SEQ ID No: 2 having a tag condensed to its N or C terminus. Such amino acid sequences also include those formed by artificial mutation of the coding nucleic acid, for example SEQ ID No: 1, SEQ ID No: 7 or SEQ ID No: 11. In general, the sequence alternatives described herein for the AK according to the invention may have at least 70%, for example 72%, 74%, 76%, 78%, at least 80%, for example 81% to 84%, at least 85%, for example 86%, 87%, 88%, 89%, 90%, 91%, 92%, 93%, 94%, 95%, 96%, 97%, 98% and 99%, sequence identity to the amino acid sequence of SEQ ID No: 2 or to an amino acid sequence comprising a SEQ ID No: 2, for example a SEQ ID No: 2 having a tag condensed to its N or C terminus. The amino acid sequences described herein may have, for example, one or more than one, for example 2, 3, 4, 5, or 6, deletions. These deletions may be at one or more, such as up to 2, up to 3, up to 4, up to 5, up to 10, up to 20, up to 50 successive amino acids at at least one site, for example two sites each, three sites each, four sites each, five sites each, ten sites each. Alternatively or additionally, there may be one or more than one, for example 2, 3, 4, 5, or 6, substitutions. These substitutions may be at one or more sites, for example 2, 3, 4, 5, 6, 7, 8, 9, 10 or 20 sites, where at at least one site, for example 1, 2, 3, 4, 5, or 6 sites, one or more, for example 1 to 50, 1 to 20, 1 to 10 or 1, 2, 3, 4, 5, 6, 7, 8, 9, 10, 20 or 50, successive amino acids may be substituted. Alternatively or additionally, there may be one or more than one, for example 2, 3, 4, 5, or 6, insertions of one or more, such as up to 2, up to 3, up to 4, up to 5, up to 10 successive amino acids at at least one site, for example 2, 3, 4, 5, 6.sites. For example, in an AK according to the invention individual elements not required for its enzymatic activity and for binding to the AKA according to the invention may be removed. All these modifications are chosen such that the enzymatic activity of the AK according to the invention is substantially unaffected. The nature of this modification on the nucleic acid level is described elsewhere.

In the present context, “substantially unaffected” is to be understood as meaning that the specific AK activity of the AK according to the invention is not less than 60%, not less than 70%, not less than 80%, based on the specific AK activity of a polypeptide having the SEQ ID No: 2.

The amino acid sequence of the AKA according to the invention has at least 60%, at least 70%, at least 80%, at least 90%, at least 95% or at least 98% sequence identity to SEQ ID No: 6 or to an amino acid sequence comprising a SEQ ID No: 6, for example SEQ ID No: 4 (complete sequence from U maydis), SEQ 1D No: 14 (complete sequence from U. maydis with an MBP tag condensed to its N terminus), or SEQ ID No: 16 (C terminus of the sequence from U. maydis with an MBP tag condensed to its N terminus). Naturally, an amino acid sequence according to the invention may be condensed on its C and/or on its N terminus with a tag. Such amino acid sequences also include those formed by artificial mutation of the coding nucleic acid, for example of SEQ ID No: 3, SEQ ID No: 5, SEQ ID No: 13 or SEQ ID No: 15. In general, the sequence alternatives described herein for the AKA may have at least 60%, for example 62%, 64%, 66%, 68%, at least 70%, for example 72%, 74%, 76%, 78%, at least 80%, for example 81% to 84%, at least 85%, for example 86%, 87%, 88% 89%, 90%, 91%, 92%, 93%, 94%, 95%, 96%, 97%, 98% and 99% sequence identity to the amino acid sequence of SEQ ID No: 4 or SEQ ID No: 10. The amino acid sequences described herein may have, for example, one or more than one, for example 2, 3, 4, 5, or 6, deletions. These deletions may be at one or more, such as up to 2, up to 3, up to 4, up to 5, up to 10, up to 20, up to 50 successive amino acids at at least one site, for example two sites each, three sites each, four sites each, five sites each, ten sites each. Alternatively or additionally, there may be one or more than one, for example 2, 3, 4, 5, or 6, substitutions. These substitutions may be at one or more sites, for example 2, 3, 4, 5, 6, 7, 8, 9, 10 or 20 sites, where at at least one site, for example 2, 3, 4, 5, or 6 sites, one or more, for example 1 to 50, 1 to 20, 1 to 10 or 2, 3, 4, 5, 6, 7, 8, 9, 10, 20 or 50, successive amino acids may be substituted. Alternatively or additionally, there may be one or more than one, for example 2, 3, 4, 5, or 6, insertions of one or more, such as up to 2, up to 3, up to 4, up to 5, up to 10 successive amino acids at at least one site, for example 2, 3, 4, 5, 6 sites. For example, in an AKA according to the invention individual elements not required for interaction with an AK according to the invention may be removed. All these modifications are chosen such that interaction with an AK is substantially unaffected, i.e. the specific activity of an AK according to the invention during the interaction with the AKA according to the invention is not less than 60%, not less than 70%, not less than 80%, based on the specific AK activity of the same AK according to the invention in the presence of an AKA having the SEQ ID No: 6. The nature of this modification on the nucleic acid level is described elsewhere.

In the context of the present application, the term “% sequence identity” refers to the proportion of identical nucleotides or amino acids between two sections of a window of optimally aligned nucleic acid or amino acids. Optimum alignment of sequences for aligning a comparison window is sufficiently known and can be carried out using auxiliaries such as the local homology algorithm of Smith and Waterman (Waterman, M. S., Chapman & Hall. London, 1995), the homology alignment algorithm of Needleman and Wunsch (1970), the similarity search of Pearson and Lipman (1988), and by computer implementations of these algorithms such as GAP, BESTFIT, FASTA, and TFASTA, which are available as component of the GCG (registered trademark), Wisconsin Package (registered trademark of Accelrys Inc., San Diego, Calif.).

For the purposes of the present application, the identity between two polypeptide sequences or else nucleic acid sequences was determined by comparison with the aid of NCBI-Blast of The National Center for Biotechnology Information (National Library of Medicine Building 38A Bethesda, Md. 20894, USA).

As described in detail above, the AKs and AKAs according to the invention may, compared to the corresponding regions of naturally occurring AKs and AKAs, respectively, have deletions or amino acid substitutions, provided they at least still have the above-described functionality of an AK having the SEQ ID No: 2 and an AKA having the SEQ ID No: 6, respectively. Conservative substitutions are preferred. Such conservative substitutions comprise variations where an amino acid is replaced by another amino acid from the group below:
1. Small aliphatic, non-polar or slightly polar residues: Ala, Ser, Thr, Pro and Gly;
2. Polar, negatively charged residues and their amides: Asp, Asn, Glu and Gln;
3. Polar, positively charged residues: His, Arg and Lys;
4. Large aliphatic, non-polar residues: Met, Leu, Ile, Val and Cys; and
5. Aromatic residues: Phe, Tyr and Trp.

The list below shows preferred conservative substitutions:

| Original Residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

One aspect of the present invention relates to a process for identifying fungicides, comprising (a) bringing a combination of a polypeptide having the biological activity of an aurora kinase (AK) and a polypeptide having the biological function of an aurora kinase activator (AKA) into contact with a chemical compound or a mixture of chemical compounds, (b) comparing the biological activity of the AK from the combination of (a) in the presence of the chemical compound or the mixture of chemical compounds to the biological activity of the AK from the combination of (a) in the absence of the chemical compound or the mixture of compounds, and, optionally, (c) determining the chemical compound from a mixture which inhibits the biological activity of the AK; where this polypeptide having the biological activity of an AK comprises (i) an amino acid sequence SEQ ID No: 2; or (ii) an amino acid sequence which has at least 80%, at least 85%, at least 90%, at least 95% and in particular at least 98% identity to the amino acid sequence SEQ ID No: 2; and where a polypeptide having the biological function of an AKA comprises (I) an amino acid sequence SEQ ID No: 6; or (II) an amino acid sequence which has at least 80%, at least 85%, at least 90%, at least 95% and in particular at least 98% identity to the amino acid sequence SEQ ID No: 6.

In one embodiment, the ratio of AK to AKA in step a) of the process according to the invention for identifying fungicides is from 2:1 to 1:10, from 2:1 to 1:5 and preferably from 1:1 to 1:5, based on the concentration of the AK and the AKA in the combination.

In a further embodiment, the concentration of the AK in the process according to the invention is at least 0.5 ng/μl, at least 1 ng/μl, at least 2 ng/μl.

In a further embodiment, the combination of AK and AKA in step a) of the process according to the invention is present in solution, preferably in aqueous solution, i.e. in this example the combination according to the invention is a solution comprising an AK according to the invention and an AKA according to the invention.

In a further embodiment, a polypeptide having the biological activity of an AK comprises an amino acid sequence SEQ ID No: 12, or an amino acid sequence having at least 80%, at least 85%, at least 90%, at least 95% and in particular at least 98% identity to the amino acid sequence SEQ ID No: 12. In a further embodiment, a polypeptide having the biological activity of an AK consists of an amino acid sequence SEQ ID No: 12.

In a further embodiment, a polypeptide having the biological function of an AKA comprises an amino acid sequence SEQ ID No: 4, SEQ ID No: 14 or SEQ ID No: 16, or an amino acid sequence having at least 80%, at least 85%, at least 90%, at least 95% and in particular at least 98% identity to one of the amino acid sequences SEQ ID No: 4, SEQ ID No: 14 or SEQ ID No: 16. In a further embodiment, a polypeptide having the biological activity of an AKA consists of an amino acid sequence SEQ ID No: 4, SEQ ID No: 14 or SEQ ID No: 16.

In a further embodiment, the comparison of the biological activity of the AK is carried out in the presence or absence of a chemical compound or the mixture of chemical compounds with the aid of a radioactive assay such as, for example, described in Muller, P., et al. (2003, Eukaryot Cell, 2(6), 1187-99), optionally followed by a quantification, for example using an AIDA image analyser, commercially available HTRF® Kinease Assays™ for serine/threonine kinases (Cat #62STOPEB. Cisbio Bioassays) or commercially available IMAP assays (Molecular Devices).

In the process described above, the fungicidal activity of the compound determined in step (c) can be tested by bringing the compound into contact with one or more fungi. Thus, for example, a fungus cell which expresses both an AK and an AKA can be brought into contact on a plate with a solution comprising the potentially fungicidal substance, or a potentially fungicidal substance is studied in a greenhouse trial or outdoor trials, where plants are initially infected with a fungus and then treated with the potentially fungicidal substance. This treatment can take place, for example, in the form of a spray solution which is sprayed onto the plants, or in the form of granules which are applied to the soil surface. A further possibility is a leaf disc test. Here, a piece is punched out from a leaf and the leaf discs, floating on a nutrient solution, are infected with an appropriate fungus and then treated with the potentially fungicidal substance.

The fungicide or the compound identified as inhibiting AK is active against at least one fungus selected from the group consisting of Blumeria species; Podosphaera species; Sphaerotheca species; Uncinula species; Gymnosporangium species; Hemileia species; Phakopsora species, for example P. pachyrhizi and P. meiborniae; Puccinia species, for example P. recondita, P. graminis, P. striiformis or P. triticina; Uromyces species; Albugo species; Bremia species; Peronospora species; Phytophthora species; Plasmopara species; Pseudoperonospora species, for example P. humuli or P. cubensis; Pythium species; Alternaria species; Cercospora species; Cladiosporum species; Cochliobolus species, for example C. sativus (conidia form: Drechslera, syn: Helminthosporium) or C. miyabeanus; Colletotrichum species; Cycloconium species; Diaporthe species; Elsinoe species; Gloeosporium species; Glomerella species; Guignardia species; Leptosphaeria species; Magnaporthe species; Mycosphaerella species; Phaeosphaeria species; Pyrenophora species; Ramularia species; Rhynchosporium species; Septoria species; Typhula species; Venturia species; Corticium species; Fusarium species; Gaeumannomyces species; Plasmodiophora species; Rhizoctonia species; Sarocladium species, for example Sarocladium oryzae; Sclerotium species, for example S. oryzae or S. rolfsii; Tapesia species; Thielaviopsis species; Alternaria species; Aspergillus species; Cladosporium species; Claviceps species; Fusarium species; Gibberella species; Monographella species; Sphacelotheca species; Tilletia species; Urocystis species; Ustilago species, such as, for example, U. nuda, U. nuda tritici, U. maydis; Aspergillus species, such as, for example, Aspergillus flavus; Botrytis species; Penicillium species, such as, for example, P. expansum or P. purpurogenum; Sclerotinia species; Verticilium species; Alternaria species; Aphanomyces species; Ascochyta species; Macrophomina species; Microdochium species; Phoma species; Phomopsis species; Phytophthora species; Typhula species; Nectria species; Monilinia species; Exobasidium species; Taphrina species; Esca species, for example Phaemoniella chlamydospora, Phaeoacremonium aleophilum or Fomitiporia mediterranea; Ganoderma species; or Helminthosporium species.

A further aspect of the present invention relates to the use of a combination of a polypeptide having the biological activity of an AK and a polypeptide having the biological function of an AKA for identifying fungicidal compounds, where the polypeptide having the biological activity of an AK comprises (i) an amino acid sequence SEQ ID No: 2; or (ii) an amino acid sequence having at least 70%, at least 80%, at least 85%, at least 90%, at least 95% and in particular at least 98% identity to the amino acid sequence from (i); and where the polypeptide having the biological function of an AKA comprises (I) an amino acid sequence SEQ ID No: 6; or (II) an amino acid sequence having at least 60%, at least 70%, at least 75%, at least 80%, at least 85%, at least 90%, at least 95% and in particular at least 98% identity to the amino acid sequence from (I).

A further aspect of the present invention relates to a nucleic acid coding for an AKA, where this AKA comprises (I) an amino acid sequence SEQ ID No: 6; or (II) an amino acid sequence having at least 60%, at least 65%, at least 70%, at least 75%, at least 80%, at least 85%, at least 90%, at least 95% and in particular at least 98% identity to the amino acid sequence from (I). In one embodiment, a nucleic acid consists of a nucleic acid coding for an AKA having SEQ ID No: 6.

Moreover, the invention relates to a nucleic acid comprising a nucleotide sequence coding for an AK, where this AK has (i) an amino acid sequence SEQ ID No: 2; or (ii) an amino acid sequence having at least 80%, at least 85%, at least 90%, at least 95% and in particular at least 98% identity to the amino acid sequence SEQ ID No: 2. In one embodiment, a nucleic acid consists of a nucleic acid coding for an AK having SEQ ID No: 2.

In one embodiment of this aspect, the nucleic acid is a nucleic acid of a phytopathogenic fungus such as, for example, U. maydis , i.e. in the exemplary case, the nucleic acid had been isolated from U. maydis.

In a further embodiment, the nucleic acid is a nucleic acid which codes for an AKA, where this AKA is an amino acid sequence SEQ ID No: 4, SEQ ID No: 14 or SEQ ID No: 16, or an amino acid sequence having at least 80%, at least 85%, at least 90%, at least 95% and in particular at least 98% identity to one of the amino acid sequences SEQ ID No: 4, SEQ ID No: 14 or SEQ ID No: 16.

In a further embodiment, the nucleic acid coding for the AK codes for an amino acid sequence comprising SEQ ID No: 12, in one embodiment consisting of SEQ ID No: 12. Such a coding nucleic acid sequence may comprise the sequence of SEQ ID No: 11 or consist of SEQ ID No: 11. Further embodiments of this nucleic acid according to the invention have been described elsewhere, for example in connection with the process according to the invention for identifying fungicides.

In the sense of the present application, nucleic acids may be DNA or RNA and single- or double-stranded. Nucleic acids can be synthesized chemically or be prepared by biological expression in vitro or else in vivo.

Nucleic acids can be synthesized chemically using suitably protected ribonucleoside posphoramidites and a conventional DNA/RNA synthesizer. Providers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

In the sense of the present application, the term “DNA” includes cDNA and genomic DNA.

Also included are nucleic acid-imitating molecules also known as synthetic or semi-synthetic derivatives of DNA or RNA and mixed polymers. Such nucleic acid-imitating molecules or nucleic acid derivatives include phosphorothioate nucleic acid, phosphoramidate nucleic acid, 2′-O-methoxyethyl ribonucleic acid, morpholino nucleic acid, hexitol nucleic acid (HNA) and “locked” nucleic acid (LNA) (Braasch and Corey, 2001). LNA is an RNA derivative in which the ribose ring is inhibited by a methylene bond between the 2′-oxygen and the 4′-carbon. In the context of the present application, it is also possible to use a peptide nucleic acid (PNA). Peptide nucleic acids have a backbone of repeat N-(2-aminoethyl)-O-glycine units linked by peptide bonds. The purine and pyrimidine bases are attached to the backbone via methylenecarbonyl bonds.

The nucleic acids described herein coding for an AKA code exclusively for an AKA capable of activating an AK, for example the AK according to the invention described herein. This applies, for example, to the AKAs encoded by SEQ ID No: 5, SEQ ID No: 3, SEQ ID No: 13 and SEQ ID No: 15 and to all modifications described elsewhere and below of the AKAs encoded by these nucleotide sequences. The nucleic acids described herein include synthetic nucleotide sequences, for example those prepared by site-specific mutagenesis of SEQ ID No: 5, SEQ ID No: 3, SEQ ID No: 13 or SEQ ID No: 15. In general, the nucleotide sequences described herein may have at least 70%, for example 72%, 74%, 76%, 78%, at least 80%, for example 81% to 84%, at least 85%, for example 86%, 87%, 88%, 89%, 90%, 91%, 92%, 93%, 94%, 95%, 96%, 97%, 98% and 99% sequence identity to the nucleotide sequence SEQ ID No: 5, SEQ ID No: 3, SEQ ID No: 13 or SEQ ID No: 15. In principle, sequence identity is calculated in a manner similar to that described above. Modifications at the nucleotide sequences disclosed herein have already been described elsewhere and also include one or more than one, for example 2, 3, 4, 5, or 6, deletions, for example of a multiple of 3 successive nucleotides at at least one site, one or more than one, for example 2, 3, 4, 5, or 6, substitutions of individual or more, for example 1 to 150, 1 to 60, 1 to 30, successive nucleotides at at least one site or one or more than one, for example 2, 3, 4, 5, or 6, insertions, for example of a multiple of 3 successive nucleotides at at least one site. It is possible, for example, to remove nucleic acid sections which code for individual elements in an AKA and which are not part of the sequence required for its binding to the AK. All these modifications are chosen such that the binding of the AKA to the AK according to the invention is not substantially affected. Further modifications include modifications to or insertion of a particular restriction enzyme cleavage site, removal of DNA to shorten the sequence, exchange of nucleotides for codon optimation or addition of further sequences or functional elements. Techniques for preparing such variants are generally known (see, for example, J. F. Sambrook, D. W. Russell, and N. Irwin, 2000).

By way of example, modifications to nucleic acids may be generated by techniques known to the person skilled in the art, such as “Site Directed Mutagenesis”, “Error Prone PCR”, “DNA shuffling” (Nature 370,1994, pp. 389-391) or “Staggered Extension Process” (Nature Biotechnol. 16,1998, pp. 258-261).

The nucleic acid described herein may comprise a number of nucleotide sequences, inter alia a nucleotide sequence selected from the group consisting of (a) the nucleotide sequence according to SEQ ID No: 5, for example SEQ ID. No: 3, SEQ ID No: 13 or SEQ ID No: 15; (b) a fragment consisting of at least 15 to 90 successive nucleotides of the nucleotide sequence from (a) coding for a peptide or at least 91 successive nucleotides of the nucleotide sequence from (a) coding for a polypeptide; (c) nucleotide sequences which hybridize with the sequences defined under a) or b) at a hybridization temperature of 20° C. to 65° C.; (d) nucleotide sequences having at least 60%, at least 65%, at least 70%, at least 75%, at least 80%, at least 85%, at least 90%, at least 95% and in particular at least 98% identity to the sequences defined under a) to c), where the polypeptides encoded by the nucleic acid have an AKA function, (e) nucleotide sequences complementary to the sequences defined under a) to d); and (f) nucleotide sequences which, as a result of the degeneration of the genetic code, code for the same amino acid sequence as the sequences defined under a) to e).

Exemplary polynucleotides are nucleotide sequences of SEQ ID No: 3 or SEQ ID No: 5. In other examples, the polypeptide consists of the amino acid sequence of SEQ ID No: 13 or SEQ ID No:15.

Nucleic acid fragments having at least 15 successive nucleotides of the nucleotide sequence from SEQ ID No: 5, for example SEQ ID No: 3, SEQ ID No: 13 or SEQ ID No: 15, preferably SEQ ID No: 3 or SEQ ID No: 5, are selected such that they can be used as specific primers or samples for the amplification or the detection of sequences of the AKA described herein, for example fragments of 15 to 40, of 18 to 40, of 20 to 40 successive nucleotides. The nucleic acid fragments can also have at least 91, at least 100, at least 120, at least 150, at least 200, at least 300, at least 400, at least 500 successive nucleotides. Nucleic acid fragments coding for polypeptides are chosen such that the AKA encoded by them maintains its ability to activate an AK, for example the AK described herein.

The term hybridization refers to the ability of a first nucleic acid strand to bind, via base pairing through hydrogen bonds, to a second nucleic acid strand, provided both nucleic acid strands are sufficiently complementary. Hybridization takes place when the two nucleic acid strands bind to each other under suitable conditions (annealing). Nucleic acid hybridization is sufficiently known to the person skilled in the art of nucleic acid manipulation techniques. The hybridization properties of a certain pair of nucleic acid strands is an indication of their complementarity. Another indication that two nucleic acid sequences are predominantly complementary is their ability to hybridize with each other under stringent conditions. Stringent hybridization conditions and stringent hybridization washing conditions in the context of nucleic acid hybridization experiments such as Southern and Northern hybridization are sequence dependent and differ depending on the chosen conditions, however, they also depend on the nucleic acids involved. Thus, for example, the melting temperatures of DNA: DNA hybrids are about 10° C. lower than those of DNA: RNA hybrids of the same length.

Stringent hybridization conditions are to be understood as meaning, for example, depending on the nucleic acid, temperatures between 20° C. and 65° C., in an aqueous buffer solution having a concentration between 0.1 bis 5×SSC (1×SSC=0.15 M NaCl, 15 mM sodium citrate, pH 7.2) or additionally in the presence of 50% formamide, such as, for example, 42° C. in 5×SSC, 50% formamide. Advantageously, the hybridization conditions for DNA: DNA hybrids are at 0.1×SSC and temperatures between about 20° C. to 65° C., preferably between about 30° C. to 45° C.

For DNA: RNA hybrids, the hybridization conditions are advantageously at 0.1×S-SC and temperatures between about 30° C. to 65° C., preferably between about 45° C. to 55° C. These stated temperatures for the hybridization are, for example, calculated melting temperature values for a nucleic acid having a length of about 100 nucleotides and a G+C content of 50% in the absence of formamide. The experimental conditions for DNA hybridization are described in the relevant textbooks of genetics such as, for example, Sambrook et al., “Molecular Cloning”, Cold Spring Harbor Laboratory, 1989, and can be calculated using formulae known to the person skilled in the art depending, for example, on the length of the nucleic acids, the type of hybrids or the G+C content. The person skilled in the art may find further information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

An example of highly stringent washing conditions comprises washing with 0.15 M NaCl at 72° C. for about 15 minutes. An example of stringent washing conditions comprises washing with 0.2×SSC at 65° C. for 15 minutes. Often, washing under highly stringent conditions is preceded by washing under low-stringency conditions to remove sample background signals. For short samples (for example 10 to 50 nucleotides), stringent conditions typically comprise salt concentrations of less than 1.5 M, for example about 0.01 to 1.0 M, a sodium ion concentration (or other salts) at pH 7.0 to 8.3; the temperature is typically at least 30° C. and at least 60° C. for long samples (for example >50 nucleotides). Stringent conditions can also be obtained by addition of destabilizing agents such as formamide. In general, a signal/noise ratio of 2× (or higher) that obtained using a sample without reference in a certain hybridization assay indicates specific hybridization. The highly stringent conditions chosen include a temperature which corresponds to that of the melting temperature of the sample.

Accordingly, nucleic acid sequences according to the invention also include those which, under standard conditions, hydridize with SEQ ID No: 1, SEQ ID No: 3, SEQ ID No: 5, SEQ ID No: 11, SEQ ID No: 13, SEQ ID No: 15, or in each case fragments of the SEQ ID Nos having at least 15 successive nucleotides and are capable of effecting expression of a polypeptide having the activity of an AK or the function of an AKA. If the activity of an AK is switched off, the resulting transformands are at least not capable of growing.

The degeneration of the genetic code is based on the fact that some of the 20 amino acids synthesized in animal and plant cells are covered multiply by two or more of the 64 possible base triplets. Triplets coding for a certain amino acid often differ only in the third, every now and then (also) in the second base. As a consequence, these positions can be exchanged when the alternative triplets are known, for example also to achieve a codon optimization of the resulting nucleic acid for expression in particular organisms.

In a further aspect, the present application relates furthermore to an expression cassette comprising an above-described nucleic acid and a promoter.

The promoter is selected depending on the organism in which the nucleic acid is to be expressed and includes promoters which can be expressed in bacteria, fungi, viruses, plants or animals. Any nucleic acid sequence which can be used as a promoter in the abovementioned organisms may serve for this purpose, including the promoter naturally occurring in these organisms. The promoter may be constitutionally active or inducible; for example, the expression may be induced under the control of a lac promoter by IPTG (isopropyl-β-D-thiogalactopyranoside). Preferred are promoters from bacteria, bacteriophages or viruses such as lac, in particular lacZ, trp, recA, nar, T7, VHb, T3-lac, CaMV, RSV promoters.

The expression cassette may additionally comprise further regulatory elements located between the promoter and the coding nucleic acid, for example transcription activators (“enhancers”), for example the transcription activator of the tobacco mosaic virus (TMV) (described in WO 87/07644), or the “tobacco etch virus” (TEV) (Carrington and Freed, 1990, J. Virol. 64: 1590-1597), or introns such as the adh1 intron from maize or intron 1 of the actin gene from rice.

The expression cassette may furthermore comprise a transcription termination or polyadenylation sequence. Here, any appropriate sequence from bacteria, for example the nos terminator from Agrobacterium tumefaciens, viruses, for example the CaMV 35S terminator, or plants, for example a histone terminator described in EP 0 633 317, may be used.

The activity or strength of a promoter may be measured in the form of the amount of RNA it produces or the protein produced in a cell or a tissue, and be compared to another promoter, the activity of which had been determined beforehand.

A further aspect is a vector comprising a nucleic acid as described above or an expression cassette as described above.

A vector includes any nucleic acid-based agent capable of carrying and transferring genetic information such as, for example, a plasmid, cosmid, virus, autonomously replicating sequence, phage, or linear single-stranded, circular single-stranded, linear double-stranded or circular double-stranded DNA or RNA nucleotide sequence. A (recombinant) vector may be derived from any source and is capable of integration into the genome or autonomous replication.

A recombinant vector typically comprises, in 5′-3′ direction: a promoter to mediate the transcription of a nucleic acid sequence and a nucleic acid sequence to be transcribed. These elements correspond to the expression cassette disclosed herein. The recombinant vector may furthermore comprise a 3′ transcription terminator, a 3′ polyadenylation signal, other non-translated nucleic acid sequences, transit and targeting nucleic acid, selectable marker, enhancer and operators, as desired. The term 5′ UTR refers to untranslated DNA upstream, or 5′ of the coding region of a gene, 3′ UTR describes the untranslated DNA downstream, or 3′ of the coding region of a gene. Means for preparing recombinant vectors are sufficiently known. Possible vectors are, for example, pENTRTM/TEV/D-Topo® (Invitrogen, Cat #45-0228, Lot #792341), pENTRTM/SD/D-Topo®, pRU11 (Brachmann et al. Mol. Microbiol., 2001, 42, (4), 1047-1063), pDEST15, pDEST17, pDEST43 (Invitrogen).

The present application also encompasses a host cell comprising a nucleic acid according to the invention, an above-described expression cassette or the vector described above.

A host cell may be any prokaryotic or eukaryotic cell including bacteria cells, fungus cells, plant cells (including tissues and whole plants) or animal cells. In addition to the nucleic acid disclosed herein and coding for an AKA or the corresponding expression cassette or the vector, the host cell may comprise a further nucleic acid coding for the AK disclosed herein, either in a separate vector or in the same vector which also comprises the nucleic acid coding for the AKA.

Host cells from mammals include the human cell lines Hela, 293, H9, SH-EP1 Jurkat, the murine cell lines NIH3T3 and C2C12, Cos 1, Cos 7 and CV1, quail QC1-3, L cells, Syrian g baby hamster kidney (BHK) cells and Chinese hamster ovary (CHO) cells. Bacterial cells which can be used as host cells include E. coli strains such as, for example, cells derived from BL21 (such as BL21(DE3), BL21(DE3)PlysS, BL21(DE3)RIL, BL21 (DE3)PRARE) or Rosetta®, Streptomyces strains and Salmonella typhimurium cells. Fungal cells such as yeasts or insect cells, for example from Drosophila S2 and Spodoptera Sf9 cells, or plant cells may also be employed. Appropriate culture media and conditions for these host cells are known to the person skilled in the art.

In addition, a further aspect of the present application relates to a polypeptide encoded by the nucleic acid described above.

Exemplary polypeptides include the amino acid sequence of SEQ ID No: 4 or SEQ ID No: 6. In other examples, the polypeptide consists of the amino acid sequence of SEQ ID No: 14 or SEQ ID No:16.

In one embodiment, a polypeptide consists of an amino acid sequence of SEQ ID No: 4, SEQ ID No: 6, SEQ ID No: 14 or SEQ ID No:16. Analogously, in one embodiment a nucleic acid according to the invention consists of a nucleotide sequence of SEQ ID No: 3, SEQ ID No: 5, SEQ ID No: 13 or SEQ ID No:15. The variants described elsewhere are likewise embraced by the polypeptide according to the invention.

Exemplary polypeptides may be overexpressed and purified by a large number of processes known to the person skilled in the art. The person skilled in the art is familiar with purification processes such as, for example, chromatographic processes, gel filtration, crystallization or dialysis. A simple purification of fusion polypeptides according to the invention having a tag such as an MBP tag can be carried out by affinity chromatography. After purification, a tag may optionally be removed. For example, an appropriate tag, provided it is attached to the AKA polypeptide via a linker sequence having an appropriate recognition sequence, can be cleaved off using AcTEV protease (Invitrogen, Cat. No. 12575-015). The term “purified” refers to a reduction of other compounds such as other proteins or cell parts from a solution comprising a polypeptide according to the invention having the biological function of an AKA, a polypeptide according to the invention having the biological activity of an AK or a combination of these two polypeptides, such that the amount of other compounds in relation to the amount of polypeptides according to the invention in a solution or a solid is reduced by one or more purification steps at least by a factor of 2, by a factor of 5, by a factor of 10, by a factor of 50. Preferably, after one or more purification steps the amount of other compounds is less than 10%, less than 1%, less than 0.1%, less than 0.01%, based on the amount of polypeptides according to the invention.

A further aspect of present application relates to a combination of a purified polypeptide according to the invention having the biological activity of an AK and a purified polypeptide according to the invention having the biological function of an AKA.

Accordingly, one aspect relates to a combination, comprising
a) a purified polypeptide having the biological function of an AKA encoded by a nucleic acid according to any of claims 7 to 9, and
b) a purified polypeptide having the biological activity of an AK, where the AK
   i) comprises an amino acid sequence SEQ ID No: 2; or
   ii) comprises an amino acid sequence having at least 70%, at least 80%, at least 85%, at least 90%, at least 95% and in particular at least 98% identity to the amino acid sequence of i).

The embodiments described for other aspects of the present invention, in particular the process for identifying fungicides, can also be applied to the combination according to the invention.

In one embodiment, a purified polypeptide having the biological function of an AKA comprises an amino acid sequence selected from the group consisting of
I) an amino acid sequence SEQ ID No: 6; or
II) an amino acid sequence having at least 60%, at least 70%, at least 75%, at least 80%, at least 85%, at least 90%, at least 95% and in particular at least 98% identity to the amino acid sequence of I).

Examples of an AKA comprising an amino acid sequence selected from SEQ ID No: 6 are the amino acid sequences SEQ ID No: 4, SEQ ID No: 14 and SEQ ID No: 16.

In a further embodiment, the combination of AK and AKA according to the invention consists of solids. It can be present, for example, in the form of lyophilized AK and AKA.

Further embodiments relate to combinations of AK and AKA in the form of solutions, emulsions or suspensions.

In a further embodiment, the combination of AK and AKA is present as a solution, preferably as an aqueous solution.

In a further embodiment, the concentration of the AK in a combination in form of a preferably aqueous solution is at least 0.5 ng/μl, at least 1 ng/μl, at least 2 ng/μl.

In a further embodiment, the ratio of AK to AKA is from 2:1 to 1:10, from 2:1 to 1:5 and preferably from 1:1 to 1:5, based on the concentration of the AK and the AKA in the combination.

A combination of AK and AKA may additionally comprise salts such as NaCl, divalent metal salts such as Mg salts (for example MgCl₂) or divalent Mn salts (for example MnCl₂) and/or buffer substances such as TRIS, HEPES, HEPPS, MOPS or MES.

A further aspect of the present application relates to antibodies capable of specifically binding to an AKA according to the invention. The person skilled in the art is aware of how to prepare antibodies by generally known processes.

The term “antibody” comprises, for example, polyclonal or monoclonal antibodies. It also includes their derivatives or fragments which have maintained their binding specificity. Derivatives or fragments include, inter alia, Fab or Fab′ fragments, Fd, F(ab′)2, Fv or scFv fragments (see for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999). The term “antibody” also comprises embodiments such as chimera (human constant domain, non-human variable domain), single-chain antibodies and humanized antibodies (a human antibody which contains non-human CDRs).

Techniques for preparing antibodies are well known and described, for example, in Harlow and Lane (1988) and (1999), loc. cit. Accordingly, antibodies can be prepared via peptidomimetics. Furthermore, techniques described for the preparation of single-chain antibodies (see for example U.S. Pat. No. 4,946,778) can be adapted to prepare single-chain antibodies which specifically bind the polypeptides according to the invention. Transgenic animals or plants (see, for example, U.S. Pat. No. 6,080,560) can be employed for expressing the antibodies according to the invention. Any technique which provides antibodies by continuous cell cultures can be used to prepare monoclonal antibodies (see for example Harlow and Lane (1988) and (1999), loc. cit.). This includes the hybridoma technique (Kohler and Milstein Nature 256 (1975), 495-497) and the trioma technique.

A further aspect relates to a process for preparing a combination of AK and AKA according to the invention, which comprises
a) expressing an AKA of a nucleic acid coding for an AKA according to the invention;
b) expressing an AK of a nucleic acid coding for an AK according to the invention; and
c) obtaining the AK and AKA polypeptides from a) and b).

In one embodiment, the nucleic acids from a) and b) are present in the same host cell, i.e. a host cell comprises both at least one nucleic acid coding for an AKA according to the invention and at least one nucleic acid coding for an AK according to the invention. Here, a host cell may either contain a vector comprising both nucleic acids, optionally integrated into an expression construct described elsewhere, or two vectors each comprising one nucleic acid or each comprising one expression construct, for example.

If host cells are used under a) and b), the AK and/or AKA polypeptides are obtained by lysis of the cells. The term lysis comprises both chemical (for example by lysozyme) and mechanical destruction (for example ultrasound) of cells. After lysis, the polypeptides according to the invention may optionally be purified further by standard processes such as, for example, affinity chromatography. In this situation, the polypeptides according to the invention are co-purified. Surprisingly, the co-purification leads to a higher activity of the AK compared to the same AK to which the AKA had only been added after the purification. The increase in activity is, for example, by a factor of 3, 6, 8, 10.

In a further embodiment, the nucleic acids from a) and b) are expressed separately in host cells, i.e. a host cell contains a vector comprising a nucleic acid coding for an AK according to the invention, or an expression construct comprising such a nucleic acid, and another host cell contains a vector comprising a nucleic acid coding for an AKA according to the invention, or an expression construct comprising such a nucleic acid. In one embodiment, the two host cells expressing AK and AKA can already be present in a mixture during the expression (steps a) and b)), i.e. AK and AKA are obtained from previously combined host cells. Alternatively, the host cells can also be combined after expression and prior to isolation, for example by purification. After expression of the polypeptides according to the invention, the host cells can, for example, be lysed together in one medium (step c)) to carry out optional purification of the polypeptides according to the invention as a co-purification, or lysis and subsequent optional purification of the AK polypeptides and AKA polypeptides according to the invention can be carried out separately, and the two polypeptides are only combined afterwards.

The person skilled in the art is aware that steps a) and b) include the cultivation of host cells under conditions which ensure expression of the nucleic acid according to the invention.

In a further embodiment, the expression is carried out in an in vitro system.

Isolation of AK and AKA polypeptides can take place, for example, in an in vitro system from Applied Biosystems (Cat#AM 1200; Product Name: Retic Lysate IVY™ Kit) (Kozak; Nuc. Acid Res. (1990); 18; 2828) or an in vitro system from Invitrogen (Cat#K9901-00; Product Name: Expressway™ Mini Cell-Free Expression System) (Savasaki et al.; PNAS (2002); 99; 14652-14657).

Purification of the polypeptides according to the invention can be carried out by standard methods such as, for example, affinity chromatography or gel electrophoresis, as described in the present application.

The combination of AK and AKA prepared by this process can be used, for example, as a combination in a process according to the invention for identifying fungicides.

A further aspect of the present invention is the provision of a kit.

A kit comprises a polypeptide according to the invention having the biological activity of an AK and a polypeptide according to the invention having the biological function of an AKA.

The polypeptides according to the invention can be present in a kit separated from one another in isolated containers or in combination in one container. Preferably, the polypeptides according to the invention are present in combination. As already described in the application, the combimation can be present in solution or as solids. Furthermore, it may be in the form of a “ready for use” solution or a “stock” solution concentrated 2×, 5×, 10×, 20×, 50× or 100×. Furthermore, a kit may comprise instructions for use on how to carry out a process according to the invention for identifying fungicides.

### General Methods

Assays for Determining the Activity of Aurora Kinases:

Assay 1: Radioactive activity determination according to Muller, P. et al. (2003), Eukaryot Cell, 2(6), 1187-1199. Substrate: myelin protein. Subsequent quantification may be by an AIDA image analyser (Version 4.13.023).

To start the activity test, 2× Mastermix and enzyme solution were mixed in 1× kinase buffer+1 mM DTT in a ratio of 1:1. The measurement was at RT over 45 min. The same volume of SDS sample buffer was then added, and the mixture was incubated at 95° C. for 5 min.

2× Mastermix

100 ng/μl of basic myelin protein

100 μM ATP

0.08 μCi/μl [γ-³²P]ATP

1 mM DTT

in 1× kinase buffer

10× Kinase buffer

500 mM Hepes pH 7.5

100 mM MgCl₂

10 mM EGTA

0.1% Brij-35

Assay 2: The activity of kinases can also be determined using 3 different substrates of the commercially available HTRF® Kinease Assays™ for serine/threonine kinases (Cat #62ST0PEB. Cisbio Bioassays).

Assay 3: For the commercially available IMAP Assay (Molecular Devices), 2 substrates for human aurora kinases (Kemptide, Cat #R7331, sequence 5TAMRA-LRRASLG-OH and PKAtide, Cat #R7250, Lot #130188, sequence 5FAM-GRTGRRNSI-NH2) may be purchased commercially. The assay was carried out in accordance with the instructions of the manufacturer.

IMAP Progressive Binding Reagent: Cat #R7284, Lot #143859

IMAP Progressive Binding Buffer A: Cat #R7285, Lot #42898

IMAP 5× Reaction Buffer: Cat #R7206, Lot #33060

### IMAP Fluorescence Polarization Assay (Assay 3)

The IMAP fluorescence polarization assay (Molecular Devices) is based on the specific high-affinity interaction between phosphate groups and trivalent metal ions. The kinase reaction phospholrylates a fluorescent substrate which is then specifically bonded by the metal beads present in the stop solution. This interaction increases the fluorescence polarization, which allows kinase activity to be determined. The assay was carried out in accordance with the instructions of the manufacturer as follows:

1 Vol. of inhibitor in 4% DMSO or 4% DMSO

1 Vol. of enzyme solution

2 Vol. of ATP/substrate

12 Vol. of stop solution

30 min incubation at RT

Enzyme solution: for example SEQ ID NO: 12 and SEQ ID NO: 16 (1:1) in Complete Reaction Buffer ATP/substrate: 70 μM ATP/200 nM FAM-PKAtide or TAMRA-Kemptide in Complete Reaction Buffer

Complete Reaction Buffer

1 Vol. of 5× IMAP Reaction Buffer (Molecular Devices)

4 Vol. of H₂O

1 mM DTT

0.01% Tween-20

Stop Solution

1/400 Vol. of Progressive Binding Reagent (Molecular Devices) in Progressive Binding Buffer A (Molecular Devices)

Fluorescence polarization was measured in a Tecan Ultra microtitre plate fluorimeter using the following measurement parameters:

Excitation wavelength 485 nm

Emission wavelength 535 nm

Integration time 40 μs

Number of measurements 3

Amplification (gain) 82

z-Position 8800 μm

A further assay which can be used to determine kinase activity is described, for example, in Bhat et al. (2003, JBC Paper, 278, 45937-45945).

### Affinity Chromatography for Purification of AK or AKA According to the Invention

In a suitable binding buffer solution, for example TRIS, HEPES or HEPPS buffer having a pH of about 6.5 to about 8.5 which may optionally comprise added salts such as NaCl and/or complex formers such as EDTA and/or reducing agents such as DTT and/or further auxiliary substances, a sample comprising fusion proteins to be purified, for example SEQ ID NO: 16, SEQ ID NO: 14 or SEQ ID NO: 12, is brought into contact with a stationary phase. It is known to the person skilled in the art that depending on the tag used different buffers comprising different auxiliary substances are expedient. Thus, for example, for a purification of fusion proteins having aHis tag, imidazole is added to the buffer solution. In contrast, in a purification based on an MBP tag, preference is given to using TRIS buffer comprising NaCl, EDTA and DTT. In one embodiment, a 10 to 100 mM TRIS buffer comprising 100 to 300 mM NaCl, 0.5 to 2 mM EDTA and 0.5 to 2 mM DTT is used: Stationary phases for affinity chromatography can lie purchased laregly commercially, for example MBPTrap (GE) for purifying MBP fusion proteins or Ni-NTA-Spin-Columns (Qiagen) for purifying His tag fusion proteins or GSTrap-Columns (GE) for purifying GST fusion proteins.

The fusion proteins to be purified bind to the stationary phase, and not specifically binding impurities are washed from the stationary phase using a wash buffer which in some cases may also consist of excess binding buffer. For MBP affinity chromatography, the wash buffer may be identical to the binding buffer. The purified fusion proteins are then detached from the stationary phase using an elution buffer comprising a suitable eluant (for example maltose for MBP tags, imidazole for His tag, glutathione for GST tag), and the buffer is optionally changed.

### EXAMPLES

### Identification SEQ ID No: 2 and SEQ ID NO: 1

Possible binding proteins for a kinase inhibitor were isolated by affinity chromatography from a cell extract from Ustilago maydis. The stationary phase used was a compound 2 (a kinase inhibitor which is covered by the disclosure of WO 2008/107096) immobilized on NHS—(N-hydroxysuccinimide)-activated Sepharose. Standard immobilization reactions are known to the person skilled in the art. Immobilization of compound 2 may take place, for example, according to Lolli et al. (2003, Proteomics, 3, 1287-1298).

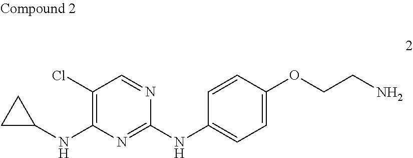

To identify possible target proteins for compound 2, 20 g of a cell pellet of Ustilago maydis were resuspended in 50 ml of Hepes lysis buffer 1+0.2 mM DTT+Complete Protease Inhibitor Cocktail tablet (Roche Art. 11873580001)+PhosStop Phosphatase Inhibitor Tablet (Roche Art. 04906837001) and homogenized in a high pressure homogenizer EmulsiFlex C 50 (5-10 passes) and centrifuged. 25 μl of supernatant were adjusted with NaCl to a final concentration of 1.15 M NaCl.

Inhibitor matrix was equilibrated with Hepes lysis buffer 1 with 1 M NaCl (matrix), 60 mg of lysate were added to 25 μl of matrix and the mixture was incubated at 4° C. for 2 h. The supernatant (=flow) was then removed, the matrix was washed twice with Hepes lysis buffer 1 comprising 1 M NaCl, once with Hepes lysis buffer 2, and potential target proteins were then eluted with 100 μl elution buffer (Hepes lysis buffer 2+0.5 mM free inhibitor (compound 2)+2.5% DMSO+10 mM ATP+20 mM MgCl₂).

| Hepes lysis buffer 1 | Hepes lysis buffer 2 |
|---|---|
| 50 mM Hepes pH 7.5 | 20 mM Hepes pH 7.5 |
| 150 mM NaCl | 150 mM NaCl |
| 0.5% Triton X-100 | 0.25% Triton X-100 |
| 10% Glycerol | 1 mM EDTA |
| 1 mM EDTA | 1 mM EGTA |
| 10 mM Na₂P₂O₇ | |

The analysis of the potential target proteins was carried out by nano HPLC/MS/MS. Processes known to the person skilled in the art are described, for example, in Knoth et al. (2009, Angewandte Chemie, 48, 7240-7245+Supplementary). The data determined were analysed for product identification using the NCBInr database (Version 20090127).

A possible target protein was identified as um10119 (SEQ ID NO: 2). The sequence obtained in this manner was entered into the U. maydis database of the MIPS (Munich Information Centre for Protein Sequences (© 2003 GSF—Forschungszentrum für Umwelt and Gesundheit, GmbH Ingolstädter Landstraβe 1, D-85764 Neuherberg)). According to MIPS, um10119 is a potential 1PL1 Ser/Thr protein kinase (MIPS/NCBI: um10119/XP_{—}756618.1). For the nucleic acid sequence (SEQ ID NO: 1) of SEQ ID NO: 2, MIPS and NCBI make differing statements concerning the presence of an intron and the resulting total length of the protein. The protein which represents um10119 according to NCBI is invalid according to MIPS and was replaced by a different, slightly shorter version.

The sequence of um10119 from MIPS was blasted in the NCBI protein database. The identified protein um10119 was classified as aurora kinase based on high sequence homology with other IPL 1/aurora kinases, for example from Puccinia graminis (homolog: aurora protein kinase, E value 3e-114, 65% identity, 79% similar amino acids), S. pombe (homolog: aurora B/Ark1, E value 6e-99, 59% identity, 77% similar amino acids) and man (homolog: aurora A, E value 3e-89, 60% identity, 75% similar amino acids).

It is known that aurora kinases of type B interact with INCENP (inner centromer protein) which is also required for full activity in vitro. Nevertheless, aurora kinases (AK) from S. cerevisiae and S. pombe are also active in the absence of the INCENP activator (AKA) (Leverson et al., 2002, Mol. Biol. Cell, 13, 1132-1143; Kang et al., 2001, J. Cell Biol., 155, 763-774).

### Expression and Activity of SEQ ID. NO: 2

### Cloning of SEQ ID NO: 1

DNA from Ustilago maydis was isolated by standard methods (for example Hoffmann and Winston, 1987, Gene, 57, 267-272). DNAStar Lasergene PrimerSelect was utilized for selecting oligonucleotides for PCR reactions and to calculate the annealing temperature. SEQ ID NO: 1 to be expressed was cloned using the Gateway® system (Invitrogen) according to the instructions of the manufacturer, initially in pENTRTM/TEV/D-Top® (Cat #45-0228, Lot #792341).

The following PCR conditions and primers were used:
1. 94° C.-5 min
2. 94° C.-30 sec
3. 56° C.-10 sec
4. 72° C.-3 min
5. 72° C.-6 min
   Steps 2 to 4 were repeated 33 times.

Primer for SEQ ID NO: 1:

AK forward (SEQ ID No: 7)

AK reverse (SEQ ID No: 9)

Correctly sequenced genes in pENTRTM/TEV/D-Topo were cloned by homologous recombination in pDESTMC2 with the aid of the Gateway® LR Clonase® enzyme mix (Invitrogen, Cat #11791019, Lot #1143296) according to the instructions of the manufacturer. This gave the expression vector pDESTMC2_SEQ ID NO: 11. SEQ ID NO: 11 codes for a fusion protein from SEQ ID NO: 1 and an MBP tag.

PDESTMC2_SEQ ID NO: 11 was transformed in expression cells OneShot® BL21(DE3)pLysE, chemically competent E. coli Invitrogen (Cat # C656503, Carlsbad, Calif., USA), according to the instructions of the manufacturer.

Expression and Purification of SEQ ID NO: 12 (MBP-AK Fusion Protein)

From a preculture of BL12(DE3)pLysE with MBP fusion protein, 1 l of LB liquid medium (+0.2% glucose, 100 μg/ml ampicillin, 34 μg/ml chloramphenicol) was seeded and cultivated at 37° C. up to an OD600 of about 0.4. At 16° C., the mixture was then shaken until an OD600 of about 0.7 had been reached, and the expression was then induced using 0.4 mM IPTG. Expression was at 16° C. over 20 h.

For work-up, the cells were resuspended in 10 ml/l of expression culture buffer A+87.5 U/ml of DNAse I, 95 μg/ml of lysozyme, 21 mM MgCl₂ and Complete Protease Inhibitor Cocktail (Roche Art. 11873580001) and disrupted by sonification. The lysate was centrifuged and the supernatant was filtered.

The supernatant clarified in this manner was loaded onto an MBP Trap HP affinity column at 1 ml/min. The column was then washed with buffer A until no more protein was detected by UV measurement. Bound protein was eluted in 0.75 ml fractions with buffer B. Protein-comprising elution fractions were concentrated. The concentrated protein solution was loaded onto a gel filtration column (HiLoad 16/60 Superdex 200, GEHealthcare Art. 17-1069-01) and the proteins were eluted with buffer A+5% glycerol in 2 ml fractions for further purification. Elution fractions having one and the same protein signal for monomeric fusion protein in the elution profile were combined and concentrated to about 1-2 ml, 20% glycerol was added (final concentration), the mixture was divided into aliquots, shock-frozen in liquid nitrogen and stored at −80° C.

| Buffer A | Buffer B |
|---|---|
| 20 mM Tris-Cl pH 7.5 | Buffer A + 10 mM maltose |
| 200 mM NaCl | |
| 1 mM EDTA | |
| 1 mM DTT | |

Activity Determination of SEQ ID NO: 12 without AKA

The activity of SEQ ID NO: 12 can be determined in different ways as described under General Methods. The method used was the radioactive assay according to Muller as described under General Methods.

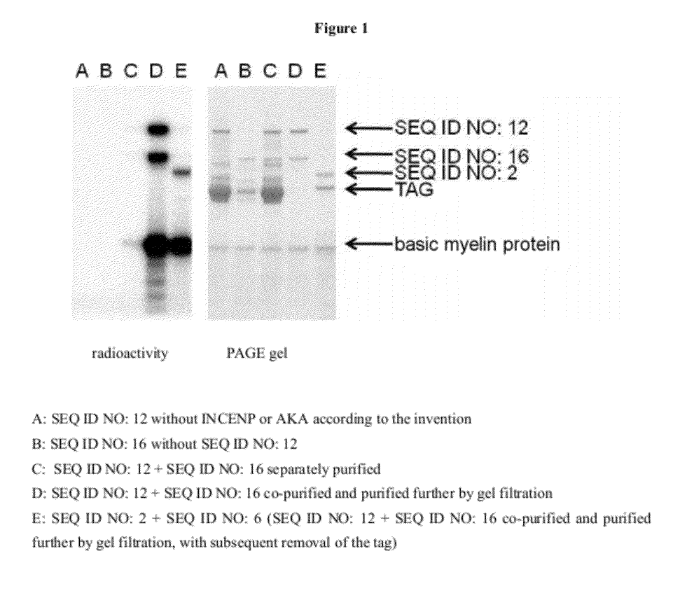

No AK activity was found. Neither the basic myelin protein (substrate) nor HistonH3 (substrate; not shown) was phosphorylated by SEQ ID NO: 12 (see FIG. 1A).

Assay 2: the activity of the AK was measured with all of the 3 different substrates of the commercially available HTRF® Kinease Assays™ for serine/threonine kinases (Cat #62ST0PEB. Cisbio Bioassays). None of the 3 substrates was phosphorylated by AK.

Assay 3: None of the two commercially available substrates (Kemptide, Cat #R7331, sequence STAMRA-LRRASLG-OH and PKAtide, Cat #R7250, Lot #130188, sequence 5FAM-GRTGRRNSI-NH2) was phosphorylated by the AK.

A radioactive Assay 1 according to Müller (2003, Eukaryot. Cell, 2, 1187-1199) with SEQ ID NO: 12 and both the INCENP sequence from S. pombe (Leverson et al., 2002, Mol. Biol. Cell, 13, 1132-1143) and the human GST-INCENP (CAT#12-534, Millipore) was then carried out.

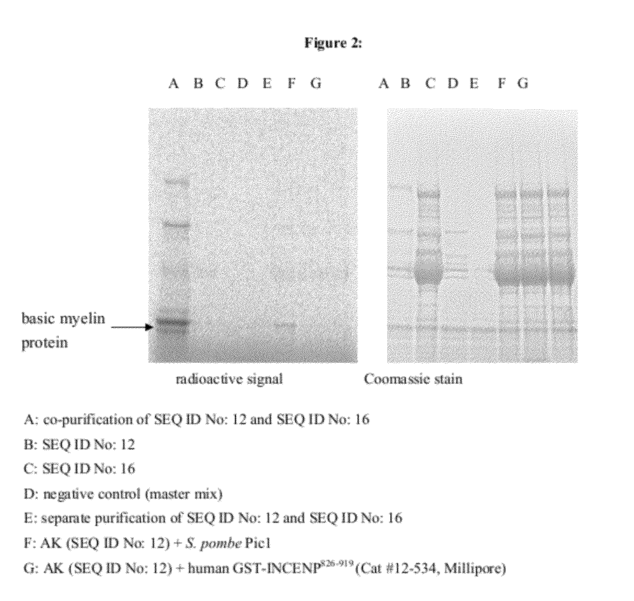

Likewise, no aurora kinase activity was found (see FIGS. 2C and 2D).

### Identification SEQ ID No: 3 (AKA)

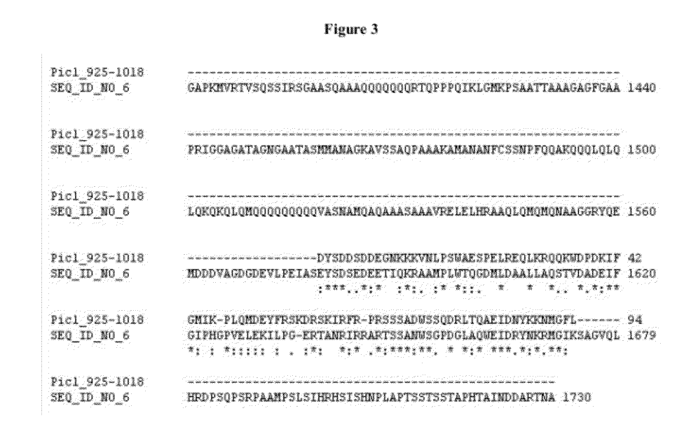

Although activity tests of proteins having the SEQ ID NO: 12 showed no aurora kinase activity in the presence of the INCENP activator from S. pombe, the sequence of this activator was blasted against the genome of U. maydis by means of MIPS (MIPS Ustilago Maydis database). No high-probability hits were found. Only in a small region at the C terminus, the sequence um03367 (postulated protein) having an E value of 2*10⁻¹⁴ showed 38% identity (FIG. 3).

### Cloning of SEQ ID NO: 5

DNA from U. maydis was isolated by standard methods (for example Hoffman and Winston, 1987, Gene, 57, 267-272). DNAStar Lasergene PrimerSelect was utilized for selecting oligonucleotides for PCR reactions and to calculate the annealing temperature. The nucleic acid sequence (SEQ ID NO: 5) corresponding to amino acids 1424 to 1575 from SEQ ID NO: 6 was cloned using the Gateway® system (Invitrogen) according to the instructions of the manufacturer, initially in pENTRTM/TEV/D-Topo® (Cat #45-0228, Lot #792341).

The following PCR conditions and primers were used:
1) 95° C.-4 min
2) 95° C.-30 sec
3) 58° C.-30 sec
4) 72° C.-1 min
5) 72° C.-10 min
6) Steps 7 to 9 were repeated 30 times.

Primers for SEQ ID NO: 5:
AKA forward (SEQ ID NO: 8)
AKA reverse (SEQ ID NO: 10)

Correctly sequenced genes in pENTRTM/TEV/D-Topo were cloned by homologous recombination in pDESTMC2 with the aid of the Gateway® LR Clonase® enzyme mix (Invitrogen, Cat #11791019, Lot #1143296) according to the instructions of the manufacturer. This gave the expression vector pDESTMC2_SEQ ID NO: 15.

PDESTMC2_SEQ ID NO: 15 was transformed in expression cells OneShot® BL21(DE3)pLysE, chemically competent E. coil lnvitrogen (Cat # C656503, Carlsbad, Calif., USA), according to the instructions of the manufacturer.

### Expression and Purification of SEQ ID NO: 16 (MBP-AKA Fusion Protein)

Expression and purification were carried out analogously to the purification of SEQ ID NO: 12 as described in paragraphs [0140] to [0142].

### Expression and Co-Purification of SEQ ID NO: 14 and SEQ ID NO: 16

Expression of the two polypeptides was in each case in OneShot® BL21(DE3)pLysE- using the MBP-AK fusion protein or in OneShot® BL21(DE3)pLysE- using the MBP-AKA fusion protein as described above. Prior to lysis, the two cell cultures were combined and then purified as described in paragraphs [0141] and [0142].

### Aurora Kinase Activity of SEQ ID NO: 12 in the Presence of SEQ ID NO: 16

As already shown in FIG. 1B, no AK activity of SEQ ID NO: 16 was found in the absence of SEQ ID NO: 12. As likewise shown (FIG. 1A), SEQ ID NO: 12, too, does not display any AK activity in the absence of SEQ ID NO: 16. The presence of Pic1 from S. pombe likewise did not result in any AK activity of SEQ ID NO: 12.

To start the activity test, 2× Mastermix and enzyme solution were mixed in 1× kinase buffer+1 mM DTT in a ratio of 1:1. The measurement was carried out at RT over 45 min. The same volume of SDS sample buffer was then added and the mixture was incubated at 95° C. for 5 min. Separation on a 4-12% Bis-Tris gel (Invitrogen) was carried out at 200 V in 1× MES buffer for 35 min. The gel was stained with Coomassie and exposed to an X-ray film for 50 min. The detection of the signals was with the aid of a Typhoon Trio 9500 scanner (Amersham Biosciences).

However, surprisingly, it could be demonstrated that SEQ ID NO: 12 in the presence of SEQ ID NO: 16 shows AK activity. In addition, it was found that, surprisingly, co-purification of the two proteins results in a higher AK activity of SEQ ID NO: 12 than the isolated purification of the two proteins and subsequent combination of these two proteins (see FIGS. 1C and D and FIGS. 2B and A).

Cleavage of the MBP Tag from SEQ ID NO: 12 and SEQ ID NO: 16

Cleavage of the MBP tag was carried out using, for example, AcTEV protease (Invitrogen, Cat #12575-015) according to the instructions of the manufacturer at 4° C. This gave SEQ ID NO: 2 and SEQ ID NO: 6.

### Aurora Kinase Activity of SEQ ID NO: 2 in the Presence of SEQ ID NO: 6

Additionally, it was shown that SEQ ID NO: 2, too, has AK activity in the presence of SEQ ID NO: 6 (AK or AKA without tag) (see FIG. 1E).

### Aurora Kinase Activity of SEQ ID NO: 12 in the Presence of SEQ ID NO: 16

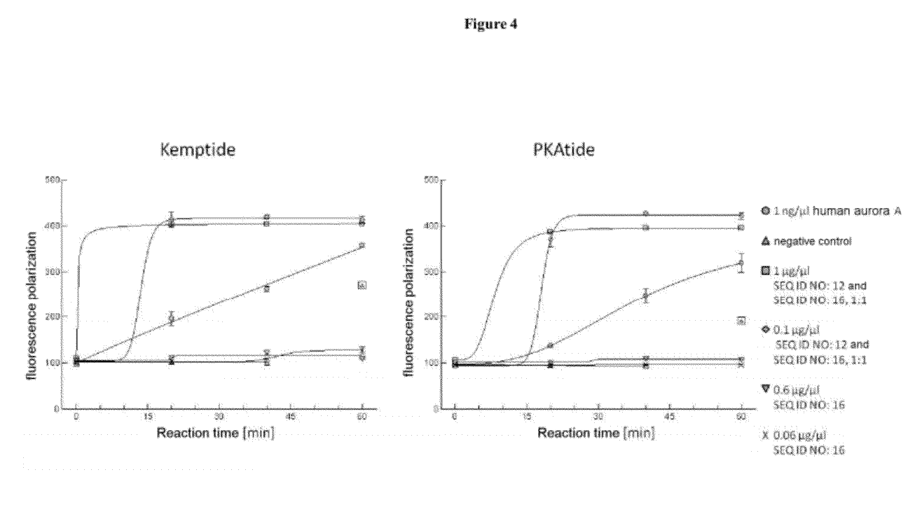

The purified complex of MBP-SEQ ID NO: 12 and MBP-SEQ ID NO: 16 was capable of phosphorylating both basic myelin protein and the substrates for human aurora kinases, PKAtide and Kemptide, in IMAP® fluorescence polarization assays (FIG. 4).

Unless indicated otherwise, the activity of the AK was determined by the IMAP method as described under General Methods.

Final enzyme concentration: 2 ng/μl, final ATP concentration 35 μM, reaction time 50 min.

Both substrates for human aurora kinase A were phosphorylated by SEQ ID NO: 12 in the presence of SEQ ID NO: 16 (see FIG. 4).

### Inhibitor Measurement

Using staurosporine (Karaman et al., 2008, Nat. Biotechnol., 26, 127-132) and compound 1

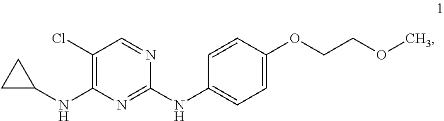

which, like compound 2, falls under the disclosure of WO 2008/107096, it was examined whether inhibition of the complex of SEQ ID NO: 12 and SEQ ID NO: 16 is also possible using compounds other than compound 2. The tests were carried out using the 1MAP assay as described above, where additionally staurosporine or compound 1 was added to the reaction solution.

The IC₅₀ of staurosporine was 2.1 nM and the IC₅₀ of compound 1 was 8.8 nM.

ED₅₀ Determination for U. maydis

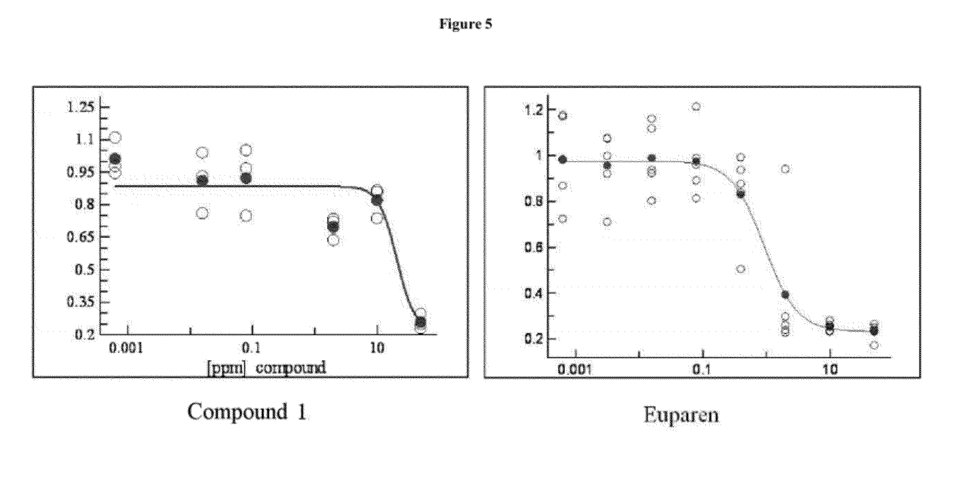

The ED50 is the concentration of a compound at which the growth of U. maydis in liquid culture is inhibited by 50%. Compounds for ED₅₀ determinations were dissolved in methanol+4 g/l of emulsifier (for example PS 16 from Bayer AG) to give 666.67 times the final concentration. 15 μl of each compound at 7 concentrations are dried at. Liquid cultures of U. maydis in Difco potato dextrose broth (BD, Cat No #254920) were adjusted to OD₆₀₀˜0.1 and in each case 200 μl of this suspension were added. The cultures were incubated at 20° C., 600 rpm and 80% atmospheric humidity for 3 days. The OD was then determined at 600 nm and plotted against the inhibitor concentration of compound 1. The positive control used was Euparen (dichlofluanid; CAS 1085-98-9), the negative control used was methanol+PS 16 without inhibitor. The ED₅₀ was determined from the OD₆₀₀ measured (FIG. 5). The ED₅₀ of compound 1 was determined as 21.8 ppm, the ED₅₀ of dichlofluanid was determined as 1 ppm.

1. Process for identifying fungicides, comprising
(a) bringing a combination of a polypeptide having the biological activity of an aurora kinase (AK) and a polypeptide having the biological function of an aurora kinase activator (AKA) into contact with a chemical compound or a mixture of chemical compounds,
(b) comparing the biological activity of the AK from the combination of (a) in the presence of the chemical compound or the mixture of chemical compounds to the biological activity of the AK from the combination of (a) in the absence of the chemical compound or the mixture of compounds, and, optionally,
(c) determining the chemical compound from a mixture which inhibits the biological activity of the AK;
where this polypeptide having the biological activity of an AK comprises
i) an amino acid sequence SEQ ID No: 2; or
ii) an amino acid sequence which has at least 80%, at least 85%, at least 90%, at least 95% and in particular at least 98% identity to the amino acid sequence SEQ ID No: 2;
and where a polypeptide having the biological function of an AKA comprises
I) an amino acid sequence SEQ ID No: 6; or
II) an amino acid sequence which has at least 80%, at least 85%, at least 90%, at least 95% and in particular at least 98% identity to the amino acid sequence SEQ ID No: 6.

2. Process according to claim 1, wherein the fungicidal activity of the compound identified in step (c) is tested by bringing it into contact with one or more fungi.

3. Process according to claim 1 or 2, wherein the ratio of a polypeptide having the biological activity of an AK and a polypeptide having the biological function of an AKA is from 2:1 to 1:10, preferably from 2:1 to 1:5 and particularly preferably from 1:1 to 1:5.

4. Process according to any of claims 1 to 3, wherein the concentration of a polypeptide having the biological activity of an AK is at least 0.5 ng/μl.

5. Process according to any of claims 1 to 4, wherein the polypeptide having the biological activity of an AK comprises an amino acid sequence SEQ ID No: 12 or an amino acid sequence having at least 80%, at least 85%, at least 90%, at least 95% and in particular at least 98% identity to an amino acid sequence SEQ ID No: 12.

6. Process according to any of claims 1 to 5, wherein the polypeptide having the biological function of an AKA comprises an amino acid sequence SEQ ID No: 4, SEQ ID No: 14 or SEQ ID No: 16 or an amino acid sequence having at least 80%, at least 85%, at least 90%, at least 95% and in particular at least 98% identity to an amino acid sequence SEQ ID No: 4, SEQ ID No: 14 or SEQ ID No: 16.

7. Use of a combination of a polypeptide having the biological activity of an AK and a polypeptide having the biological function of an AKA for identifying fungicidal compounds, where the polypeptide having the biological activity of an AK comprises
i) an amino acid sequence SEQ ID No: 2; or
ii) an amino acid sequence which has at least 70%, at least 80%, at least 85%, at least 90%, at least 95% and in particular at least 98% identity to the amino acid sequence from (i);
and
where the polypeptide having the biological function of an AKA comprises
I) an amino acid sequence SEQ ID No: 6; or
II) an amino acid sequence having at least 60%, at least 70%, at least 75%, at least 80%, at least 85%, at least 90%, at least 95% and in particular at least 98% identity to the amino acid sequence SEQ ID No: 6.

8. Nucleic acid coding for an AKA, where this AKA comprises
I) an amino acid sequence SEQ ID No: 6; or
II) an amino acid sequence having at least 60%, at least 70%, at least 75%, at least 80%, at least 85%, at least 90%, at least 95% and in particular at least 98% identity to the amino acid sequence SEQ ID No: 6.

9. Nucleic acid according to claim 8, where the AKA comprises an amino acid sequence SEQ ID No: 4, SEQ ID No: 14 or SEQ ID No: 16 or an amino acid sequence having at least 80%, at least 85%, at least 90%, at least 95% and in particular at least 98% identity to one of the amino acid sequences SEQ ID No: 4, SEQ ID No: 14 or SEQ ID No: 16.

10. Nucleic acid according to claim 8 or 9, comprising a nucleotide sequence selected from the group consisting of
a) the nucleotide sequence according to SEQ ID No: 5;
b) a fragment consisting of at least 15 to 90 successive nucleotides of the nucleotide sequence from (a) coding for a peptide or at least 91 successive nucleotides of the nucleotide sequence from (a) coding for a polypeptide;
c) nucleotide sequences which hybridize with the sequences defined under a) or b) at a hybridization temperature of 20-65° C.;
d) nucleotide sequences having at least 70%, at least 75%, at least 80%, at least 85%, at least 90%, at least 95% and in particular at least 98% identity to the sequences defined under a) to c), where the polypeptides encoded by the nucleic acid have an AKA function;
e) nucleotide sequences complementary to the sequences defined under a) to d); and
f) nucleotide sequences which, as a result of the degeneration of the genetic code, code for the same amino acid sequence as the sequences defined under a) to e).

11. Expression cassette (DNA construct), comprising a nucleic acid according to claim 8 or 10 and a promoter.

12. Vector, comprising a nucleic acid according to any of claims 8 to 10, or an expression cassette (DNA construct) according to claim 11.

13. Host cell, comprising a nucleic acid according to any of claims 8 to 10, an expression cassette (DNA construct) according to claim 11 or a vector according to claim 12.

14. Polypeptide encoded by the nucleic acid of any of claims 8 to 10.

15. Polypeptide of claim 14, comprising SEQ ID No: 4, SEQ ID No: 6, SEQ ID No: 14 or SEQ ID No: 16.

16. Combination, comprising
a) a purified polypeptide having the biological function of an AKA encoded by a nucleic acid according to any of claims 7 to 9, and
b) a purified polypeptide having the biological activity of an AK, where the AK
i) comprises an amino acid sequence SEQ ID No: 2; or
ii) comprises an amino acid sequence having at least 70%, at least 80%, at least 85%, at least 90%, at least 95% and in particular at least 98% identity to the amino acid sequence of i).

17. Combination according to claim 16, where the AKA comprises an amino acid sequence selected from the group consisting of
I) an amino acid sequence SEQ ID No: 6; or
II) an amino acid sequence having at least 60%, at least 70%, at least 75%, at least 80%, at least 85%, at least 90%, at least 95% and in particular at least 98% identity to the amino acid sequence of I).

18. Antibody which binds specifically to an AKA according to claim 14 or 15.

19. Process for preparing a combination of AK and AKA according to claim 14 or 15, which comprises
a) expressing an AKA of a nucleic acid coding for the polypeptide of claim 14 or 15;
b) expressing an AK of a nucleic acid coding for an AK as described in claim 16 b); and
c) obtaining the AK and AKA polypeptides; preferably by purification, preferably co-purification, of the AKA from a) and of the AK from b).

20. Process according to claim 19, wherein the nucleic acids from a) and b) are present together in a host cell.

21. Process according to claim 19, wherein the nucleic acids from a) and b) are present in separate host cells.

22. Process according to claim 21, wherein the host cells expressing AK and AKA are present in a mixture during the expression.

23. Process according to any of claim 19, 21 or 22, wherein AK and AKA are obtained from host cells combined beforehand.

24. Process according to any of claims 1 to 4, wherein the combination of AK and AKA is prepared according to any of claims 19 to 23.

25. Kit, comprising a polypeptide according to claim 14 or 15 and a polypeptide having the biological activity of an AK comprising
i) an amino acid sequence SEQ ID No: 2; or
ii) an amino acid sequence having at least 80%, at least 85%, at least 90%, at least 95% and in particular at least 98% identity to the amino acid sequence SEQ ID No: 2.


SEQUENCE LISTING (16 sequences)

SEQ ID NO: 1 - DNA, 1428 bp, Ustilago maydis
  Aurorakinase
atggagtcgcagctcgccaacttgcacctggatggcggctcgacggcagatctcaagcaaggatccagaccgtcacatgcacaggcatcgcacggaaacagacatgttcatcctacaaactcgtcactccgacctctggcgccttccagctcaattgcgtccaacatgggtatcaatatgggtgtcaacatgcagcacttcaaccgtctagctgcctcctcaacggggctcacgaaccccaacagcattgcacaaggcatgagccgagctgcgcagcagccgctcaacggatcgagacctagcacacaacaacagcctacgcaggcacatatcaagagtcgacctcctagcgttgctggcggactgaatgctgcggctggtccttcgtatcgtccagcggcgcctgctgctcaatctaccaaacaaccagctagtgctggatcagccgtagcatcggttgatttgggacgatatgacggtggtctggaacgcgatgaagcacgcggacgtcgaggaacgatgcaattcgatccgctcacgctttcctctgccgatgctgggaaacaacatccaccaacccgcgtgtggtcgctcaaagacttcgaaatgggtcgacctcttggcaaaggcaaattcggtcgcgtgtacatggttcgcactcgcgctgcacccaacaaaggctacattatcgccctcaagtgcatgtacaaaaacgagctagtcgagaacaaggtggaaaaacaacttcgaagagagatcgagatccagatgaacctccgccatcctcatatccttcgacttcacggatacttccacgatgagggccgagtcttcctcatgatcgaatttgctggtcgtggggagctctacaaattgatgaacaaactacacgatcgaaggttcgaagaaaaggttgcagcaacctacatcgcgcagatggcagacgctctatcatacctgcacagcaagcacgtcattcatcgcgacatcaaaccagaaaaccttctattgggtatcaaaggcgatctcaagattggtgactttggatggagcgttcacgcccctggaaaccgtcgtcagactctgtgtggtaccttggactacttgccccccgagatggtaaatggagagcaacacgataaggcagtagacctgtgggcattgggcgtgctgtgttacgaatttttggaaggtgttcctccgtttgaagagctggaaaacgctcccgcaggcacgtacaagagaatcaacaacatcgacttcaagatccctcgacatttcagccccgaagctgccgatctggtaaaggcactgctaaagaagaaaccagacgacaggctacccttgaccaaagtgctgaggcatccgtggatcatgaagtacgatccagatgcttgtcgtcgtgcgtctcgtggcaaaattcagtaa

SEQ ID NO: 2 - protein, 475 aa, Ustilago maydis
  Aurorakinase
MESQLANLHLDGGSTADLKQGSRPSHAQASHGNRHVHPTNSSLRPLAPSSSIASNMGINMGVNMQHFNRLAASSTGLTNPNSIAQGMSRAAQQPLNGSRPSTQQQPTQAHIKSRPPSVAGGLNAAAGPSYRPAAPAAQSTKQPASAGSAVASVDLGRYDGGLERDEARGRRGTMQFDPLTLSSADAGKQHPPTRVWSLKDFEMGRPLGKGKFGRVYMVRTRAAPNKGYIIALKCMYKNELVENKVEKQLRREIEIQMNLRHPHILRLHGYFHDEGRVFLMIEFAGRGELYKLMNKLHDRRFEEKVAATYIAQMADALSYLHSKHVIHRDIKPENLLLGIKGDLKIGDFGWSVHAPGNRRQTLCGTLDYLPPEMVNGEQHDKAVDLWALGVLCYEFLEGVPPFEELENAPAGTYKRINNIDFKIPRHFSPEAADLVKALLKKKPDDRLPLTKVLRHPWIMKYDPDACRRASRGKIQ

SEQ ID NO: 3 - DNA, 5193 bp, Ustilago maydis
  Aurorakinaseaktivator
atgctccgcgttcggtttggtgcctgtccgcttgcattcatcctgaccatcatcactctccggatcacacccccacctgtcggctcttccagattccaattgatgcactttgtgctagtactcgcgcatcttgccagcagacgattagattcacgaccagagaaacgctgcctacacttgcatccgcctcaaatcggctcacgccatccatcctggtatccacgctcatacagcgactcggacaagctccgaccaacctcgtccgccgattcatctgtctcttttcttggtagcaaatcaatcacactcagcagccatgccgacagctcgaaccgcttctcgcaccgtgtcccggccttggactcctcggcaagcacctcggctgcgagtagtgactcaagctcaccactgaccccagaagagcgtctgctcagccacgtcaacaacatgcgcaggtgctgcgacatgtctctcttccacaagaacctccaagagcaaggtatcgattttattgcagatagccttttgcatgccaacgacatctttgccaagtacgcaacaacgtcgcgcaccgttgcctcgccgtccaagctggctggcgaactcctcaagtctcccacccgccctcgcacccgcgctcgtgatgcgccagccgcatcaggctatcctcgtggggcattcaaagtatcgattgatcccttcgtcgatgcggccgacgtgttcgacaaggaaaatgcacccttgcaaactagatcttcacgatcacgtaccgcttccactgtcgagcctcagaaaggcaagggcaaggctagagccaaagctattcatgctgtcgaaagagaagcatcgtcacctctttctcgcccgacccgtagagtgcttggcgatgtcaacacgcaaaagaatgagacaaagcaacccactccacagcccaagcccaccagcaaagaagcaacgctcaagaacattgccacagatgacgacagcatggtcattgattcttcgctccaagcgcacctctcacccgatgataccatgccaagagcttccacaccaccaccgcctcaagcgtcgcctccgagaatacttgcccttcacaagccttccaaggccactcagtccgcagcgtcactctcgggcgccatttccatcactgattcggacgaggacggctcagacggagattcggacgccgatttgcacgaagaagagacaggagggctaaacgataccgttcaacatgaagttgccgagcctacagtgacaggaaatgtcgtgtctcggagctcccgcagcgatgacaacacagcatccttccaacctgacataaacgacacgatgccagccaacgacaacaccgtgtctggtactttgattgatccgaaactcgaaagtgcccgtcaagcagcctctgtactgcttacaagcaaaattcagcagcagaaggatgcaaacgccatgaagccggtcaaacggacgcttgcatcgtacggcgcaaaaaagagcctgccagctgctgccaacaactccggtcccagcaacgtaaccaccacgatcgttcccacctcgacccccgctggtcctggcttccgttccagcttcctcaacaagagtctgcgcaaacagattgccgaccaggaagctctcgaaagcggccaagactctgactctgactcggacgacaacgaaaacgacacaggtcttgtcgccggcgccacctttgctgctatgtacaaaaaggcagccgccaacacgcaagccaatactcgcgcaaatgctgtaccagcgtcaaacagcaccactagctccaagaagcgcaagagtgacgaggttcttccagctgcgattgaggccagcttgagcgctgccgctccaccgcacaaggtgtccaagctgggtgccgcgatgatggcggccaagacacccacgtccgctcaaccatctactgccaagaaagcaccagctgccggccctgcgtccaaactggagcaattccgcaacaacctcgctcatgttgctcgcagcaccggaagcggtgcgagtgccagctcggcttctcagtcagcgtcgtccgtcacgtcgcccaccatggaacaagcaccatccgcgccgctgcccgtcgtcaagccttcggatgcggccgtcgtgccattcgctcaagaagcgtcggcactgccttccattgaaagcttgaatgcttcgcattcgaaatcctcggattctggctccgactcgacggaaaaacttacggcaggtaccaagagtggcgagtcaaccatcaaggcttctgccatcccacgatcgcccactcgctctcctcgcgccgccgccacttcaccgatgcggcctccgcctcgcaccggcagtgtgcgtaaatctccacgcaaagtggccgagcagatcaacacaagcggacaagagtcggccagatcggcttcgccggggcccaaggtgaaagaaagtatagctcttttccagtcgagcagaaacggcggtctgactgataagcacacacgtagccctaccacgcagccccgaagtctcgcagcactctttggcagtcccaagcaagcgaccaaactaccgacgctgtcggccactgccggcagcagcaccaccccggctcactctccacccaagaactggcaaggaagtcgtttaccgtttgcacaaaccttccagttgcaacagcaagcgctgcgtgaggtggacaagtctttgccgcagacaccggccgaggagacggccgacagccagcgtgtcgtctcggcagcctcgacggtcttgtccacgtccaaggctgaaacagcggagagccaattcttcgacgcccgctcgagggagaacagtgccgagccagccgatgcgccagctccagcgccattggcggcgatgaaaggcgcgacggcacctgggaccagtgcacctcaagtggtcgccgaaaaggagtgcgtcatgcttgctgaggcacccatcgttgccaacaaggcatccagcattgtgaagactagctcgctttcaccaaagaaagtggcggccaaggcgcttgcgaaaactggaccgacctcgccgaccaaggcgagcagtctccgagctgcagccaacgccacgtcgccttccaagatcaaggcgtcggcgcgcacagcacagcagcatggcggaccgggactgggaccgggaccggcaccgtctggatcgccttcgagggcttggggcattggtgaaaagatcaaagggttcctcggattgcatgcctcgacgacgatccacccatctacagcagttgctcagaccaaggtatcggcgtctggcacattccagccgcgtgccaacgggtttgccacaaacacggctcatgcgagcgcttccgaacacaagactgcggctacagctacaccggcaccttgcattcctggcgctctgcttgctagccctccgccaacggccaagtctgcgcaggtcaactcaacaccctcaagcggtgctgcttcggcgacggccaacggaaagctcaccagcattgcgcgcgccgaaatgctgcgcaagaagcaggcagaggaggaggagcgcaaggctaaggacaagcaagagcgtcgaagaatgctgatggccaagaaggaagagcgtgccaaggcggtcgcggaggaggaaatcgaaaaggagaaccgcaaacgagcgcgtgagcagaacgaccgcctcggccgttccggcatcgaagacgccagcatcgcttcgtctaacggtgcaccggctgcgattcctgctgcgctcctgggcaatattgtcgtcggtggcggtggctccgcaatttctggcagtgttcacagcggctcagctagctcaaggacaagcacagtcagcaccgttagcagcggaccgcccacgcgaccatcaagtgctctcaatggaagctatgctggtgtcaagagtcgctacatgcagggagcggtccagcagcagcagcagcagccgccgccgcagcaacaaggcgaggaaagtagtaagaagcgtcgcgtcacaaatgacaaggaaggtatagcggcaggcttgcaagtgccattgaagcagcagccggcgcctcagcaatcacaaggtgcacctagcgctcgtcctggtgcttcggtggcgacgtcgcagatcggtgcacccaagatggttcgcacagtcagccagtcgtcgatacgatctggtgctgcctcacaagctgctgcccagcaacagcaacagcaacagcgcactcagccgccgccgcagatcaagctgggcatgaagccgagtgcggcgacgacagcagcagcaggagcaggatttggagcagcgccacgcataggcggagctggtgctacagccggcaacggcgcggctactgcgtcaatgatggcgaatgctggcaaagctgtttcgtcggcgcaacccgcggcagccaaggctatggcgaatgccaacttttgctcttcgaatccgttccagcaagccaagcagcagcaactgcagctgcagttgcaaaagcagaagcagcttcaaatgcagcaacagcagcaacagcaacagcaagttgcatccaacgcgatgcaggcgcaggcggctgctagtgcggcagcggtgcgagaattagagttgcatcgtgcggcgcaattgcaaatgcagatgcagaatgcagctggtggacggtatcaagagatggacgacgatgttgcaggcgatggcgacgaggtgctgccggagattgcatcggaatactcggactcggaagacgaagagacaattcagaagcgagcggcgatgccgttgtggacgcagggcgatatgctggatgcggcgctgttggcgcaatccacggtggatgccgatgagatctttggtattccacatggtcctgttgagctagaaaaaatcttgcctggcgagcgcacagcgaaccggatccgacgagcgcgaacttcgtcggccaactggtccgggcccgacggcctcgcgcagtgggagatcgacagatacaacaaacgcatgggcatcaagagtgcaggtgtccagttacatcgcgacccatctcagccgtctcggccagccgctatgccatccctctcgatccaccgacactcgatctcgcacaacccactcgctcccacctcgtccacctcgtccactgctccacacacagccatcaacgacgacgctcgtacaaatgcttag

SEQ ID NO: 4 - protein, 1730 aa, Ustilago maydis
  Aurorakinaseaktivator
MLRVRFGACPLAFILTIITLRITPPPVGSSRFQLMHFVLVLAHLASRRLDSRPEKRCLHLHPPQIGSRHPSWYPRSYSDSDKLRPTSSADSSVSFLGSKSITLSSHADSSNRFSHRVPALDSSASTSAASSDSSSPLTPEERLLSHVNNMRRCCDMSLFHKNLQEQGIDFIADSLLHANDIFAKYATTSRTVASPSKLAGELLKSPTRPRTRARDAPAASGYPRGAFKVSIDPFVDAADVFDKENAPLQTRSSRSRTASTVEPQKGKGKARAKAIHAVEREASSPLSRPTRRVLGDVNTQKNETKQPTPQPKPTSKEATLKNIATDDDSMVIDSSLQAHLSPDDTMPRASTPPPPQASPPRILALHKPSKATQSAASLSGAISITDSDEDGSDGDSDADLHEEETGGLNDTVQHEVAEPTVTGNVVSRSSRSDDNTASFQPDINDTMPANDNTVSGTLIDPKLESARQAASVLLTSKIQQQKDANAMKPVKRTLASYGAKKSLPAAANNSGPSNVTTTIVPTSTPAGPGFRSSFLNKSLRKQIADQEALESGQDSDSDSDDNENDTGLVAGATFAAMYKKAAANTQANTRANAVPASNSTTSSKKRKSDEVLPAAIEASLSAAAPPHKVSKLGAAMMAAKTPTSAQPSTAKKAPAAGPASKLEQFRNNLAHVARSTGSGASASSASQSASSVTSPTMEQAPSAPLPVVKPSDAAVVPFAQEASALPSIESLNASHSKSSDSGSDSTEKLTAGTKSGESTIKASAIPRSPTRSPRAAATSPMRPPPRTGSVRKSPRKVAEQINTSGQESARSASPGPKVKESIALFQSSRNGGLTDKHTRSPTTQPRSLAALFGSPKQATKLPTLSATAGSSTTPAHSPPKNWQGSRLPFAQTFQLQQQALREVDKSLPQTPAEETADSQRVVSAASTVLSTSKAETAESQFFDARSRENSAEPADAPAPAPLAAMKGATAPGTSAPQVVAEKECVMLAEAPIVANKASSIVKTSSLSPKKVAAKALAKTGPTSPTKASSLRAAANATSPSKIKASARTAQQHGGPGLGPGPAPSGSPSRAWGIGEKIKGFLGLHASTTIHPSTAVAQTKVSASGTFQPRANGFATNTAHASASEHKTAATATPAPCIPGALLASPPPTAKSAQVNSTPSSGAASATANGKLTSIARAEMLRKKQAEEEERKAKDKQERRRMLMAKKEERAKAVAEEEIEKENRKRAREQNDRLGRSGIEDASIASSNGAPAAIPAALLGNIVVGGGGSAISGSVHSGSASSRTSTVSTVSSGPPTRPSSALNGSYAGVKSRYMQGAVQQQQQQPPPQQQGEESSKKRRVTNDKEGIAAGLQVPLKQQPAPQQSQGAPSARPGASVATSQIGAPKMVRTVSQSSIRSGAASQAAAQQQQQQQRTQPPPQIKLGMKPSAATTAAAGAGFGAAPRIGGAGATAGNGAATASMMANAGKAVSSAQPAAAKAMANANFCSSNPFQQAKQQQLQLQLQKQKQLQMQQQQQQQQQVASNAMQAQAAASAAAVRELELHRAAQLQMQMQNAAGGRYQEMDDDVAGDGDEVLPEIASEYSDSEDEETIQKRAAMPLWTQGDMLDAALLAQSTVDADEIFGIPHGPVELEKILPGERTANRIRRARTSSANWSGPDGLAQWEIDRYNKRMGIKSAGVQLHRDPSQPSRPAAMPSLSIHRHSISHNPLAPTSSTSSTAPHTAINDDARTNA

SEQ ID NO: 5 - DNA, 456 bp, Ustilago maydis
  Aurorakinaseaktivator (C-Terminus)
tactcggactcggaagacgaagagacaattcagaagcgagcggcgatgccgttgtggacgcagggcgatatgctggatgcggcgctgttggcgcaatccacggtggatgccgatgagatctttggtattccacatggtcctgttgagctagaaaaaatcttgcctggcgagcgcacagcgaaccggatccgacgagcgcgaacttcgtcggccaactggtccgggcccgacggcctcgcgcagtgggagatcgacagatacaacaaacgcatgggcatcaagagtgcaggtgtccagttacatcgcgacccatctcagccgtctcggccagccgctatgccatccctctcgatccaccgacactcgatctcgcacaacccactcgctcccacctcgtccacctcgtccactgctccacacacagccatcaacgacgacgctcgtacaaatgcttag

SEQ ID NO: 6 - protein, 151 aa, Ustilago maydis
  Aurorakinaseaktivator (C-Terminus)
YSDSEDEETIQKRAAMPLWTQGDMLDAALLAQSTVDADEIFGIPHGPVELEKILPGERTANRIRRARTSSANWSGPDGLAQWEIDRYNKRMGIKSAGVQLHRDPSQPSRPAAMPSLSIHRHSISHNPLAPTSSTSSTAPHTAINDDARTNA

SEQ ID NO: 7 - DNA, 26 bp, Artificial Sequence
  Nukleinsauresequenz Primer AK_forward
caccatggagtcgcagctcgccaact

SEQ ID NO: 8 - DNA, 29 bp, Artificial Sequence
  Nukleinsauresequenz Primer AKA_forward
cacctactcggactcggaagacgaagaga

SEQ ID NO: 9 - DNA, 23 bp, Artificial Sequence
  Nukleinsauresequenz Primer AK_reverse
ttactgaattttgccacgagacg

SEQ ID NO: 10 - DNA, 22 bp, Artificial Sequence
  Nukleinsauresequenz Primer AKA_reverse
ctaagcatttgtacgagcgtcg

SEQ ID NO: 11 - DNA, 2679 bp, Artificial Sequence
  Aurorakinase mit MBP-TAG
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattatcaacaagtttgtacaaaaaagcaggctccgcggccgccgtactcgaggaaaacctgtattttcagggctccttcaccatggagtcgcagctcgccaacttgcacctggatggcggctcgacggcagatctcaagcaaggatccagaccgtcacatgcacaggcatcgcacggaaacagacatgttcatcctacaaactcgtcactccgacctctggcgccttccagctcaattgcgtccaacatgggtatcaatatgggtgtcaacatgcagcacttcaaccgtctagctgcctcctcaacggggctcacgaaccccaacagcattgcacaaggcatgagccgagctgcgcagcagccgctcaacggatcgagacctagcacacaacaacagcctacgcaggcacatatcaagagtcgacctcctagcgttgctggcggactgaatgctgcggctggtccttcgtatcgtccagcggcgcctgctgctcaatctaccaaacaaccagctagtgctggatcagccgtagcatcggttgatttgggacgatatgacggtggtctggaacgcgatgaagcacgcggacgtcgaggaacgatgcaattcgatccgctcacgctttcctctgccgatgctgggaaacaacatccaccaacccgcgtgtggtcgctcaaagacttcgaaatgggtcgacctcttggcaaaggcaaattcggtcgcgtgtacatggttcgcactcgcgctgcacccaacaaaggctacattatcgccctcaagtgcatgtacaaaaacgagctagtcgagaacaaggtggaaaaacaacttcgaagagagatcgagatccagatgaacctccgccatcctcatatccttcgacttcacggatacttccacgatgagggccgagtcttcctcatgatcgaatttgctggtcgtggggagctctacaaattgatgaacaaactacacgatcgaaggttcgaagaaaaggttgcagcaacctacatcgcgcagatggcagacgctctatcatacctgcacagcaagcacgtcattcatcgcgacatcaaaccagaaaaccttctattgggtatcaaaggcgatctcaagattggtgactttggatggagcgttcacgcccctggaaaccgtcgtcagactctgtgtggtaccttggactacttgccccccgagatggtaaatggagagcaacacgataaggcagtagacctgtgggcattgggcgtgctgtgttacgaatttttggaaggtgttcctccgtttgaagagctggaaaacgctcccgcaggcacgtacaagagaatcaacaacatcgacttcaagatccctcgacatttcagccccgaagctgccgatctggtaaaggcactgctaaagaagaaaccagacgacaggctacccttgaccaaagtgctgaggcatccgtggatcatgaagtacgatccagatgcttgtcgtcgtgcgtctcgtggcaaaattcagtaa

SEQ ID NO: 12 - protein, 892 aa, Artificial Sequence
  Aurorakinase mit MBP-TAG
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISELSTSLYKKAGSAAAVLEENLYFQGSFTMESQLANLHLDGGSTADLKQGSRPSHAQASHGNRHVHPTNSSLRPLAPSSSIASNMGINMGVNMQHFNRLAASSTGLTNPNSIAQGMSRAAQQPLNGSRPSTQQQPTQAHIKSRPPSVAGGLNAAAGPSYRPAAPAAQSTKQPASAGSAVASVDLGRYDGGLERDEARGRRGTMQFDPLTLSSADAGKQHPPTRVWSLKDFEMGRPLGKGKFGRVYMVRTRAAPNKGYIIALKCMYKNELVENKVEKQLRREIEIQMNLRHPHILRLHGYFHDEGRVFLMIEFAGRGELYKLMNKLHDRRFEEKVAATYIAQMADALSYLHSKHVIHRDIKPENLLLGIKGDLKIGDFGWSVHAPGNRRQTLCGTLDYLPPEMVNGEQHDKAVDLWALGVLCYEFLEGVPPFEELENAPAGTYKRINNIDFKIPRHFSPEAADLVKALLKKKPDDRLPLTKVLRHPWIMKYDPDACRRASRGKIQ

SEQ ID NO: 13 - DNA, 6444 bp, Artificial Sequence
  Aurorakinaseaktivator mit MBP-TAG
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattatcaacaagtttgtacaaaaaagcaggctccgcggccgccgtactcgaggaaaacctgtattttcagggctccttcaccatgctccgcgttcggtttggtgcctgtccgcttgcattcatcctgaccatcatcactctccggatcacacccccacctgtcggctcttccagattccaattgatgcactttgtgctagtactcgcgcatcttgccagcagacgattagattcacgaccagagaaacgctgcctacacttgcatccgcctcaaatcggctcacgccatccatcctggtatccacgctcatacagcgactcggacaagctccgaccaacctcgtccgccgattcatctgtctcttttcttggtagcaaatcaatcacactcagcagccatgccgacagctcgaaccgcttctcgcaccgtgtcccggccttggactcctcggcaagcacctcggctgcgagtagtgactcaagctcaccactgaccccagaagagcgtctgctcagccacgtcaacaacatgcgcaggtgctgcgacatgtctctcttccacaagaacctccaagagcaaggtatcgattttattgcagatagccttttgcatgccaacgacatctttgccaagtacgcaacaacgtcgcgcaccgttgcctcgccgtccaagctggctggcgaactcctcaagtctcccacccgccctcgcacccgcgctcgtgatgcgccagccgcatcaggctatcctcgtggggcattcaaagtatcgattgatcccttcgtcgatgcggccgacgtgttcgacaaggaaaatgcacccttgcaaactagatcttcacgatcacgtaccgcttccactgtcgagcctcagaaaggcaagggcaaggctagagccaaagctattcatgctgtcgaaagagaagcatcgtcacctctttctcgcccgacccgtagagtgcttggcgatgtcaacacgcaaaagaatgagacaaagcaacccactccacagcccaagcccaccagcaaagaagcaacgctcaagaacattgccacagatgacgacagcatggtcattgattcttcgctccaagcgcacctctcacccgatgataccatgccaagagcttccacaccaccaccgcctcaagcgtcgcctccgagaatacttgcccttcacaagccttccaaggccactcagtccgcagcgtcactctcgggcgccatttccatcactgattcggacgaggacggctcagacggagattcggacgccgatttgcacgaagaagagacaggagggctaaacgataccgttcaacatgaagttgccgagcctacagtgacaggaaatgtcgtgtctcggagctcccgcagcgatgacaacacagcatccttccaacctgacataaacgacacgatgccagccaacgacaacaccgtgtctggtactttgattgatccgaaactcgaaagtgcccgtcaagcagcctctgtactgcttacaagcaaaattcagcagcagaaggatgcaaacgccatgaagccggtcaaacggacgcttgcatcgtacggcgcaaaaaagagcctgccagctgctgccaacaactccggtcccagcaacgtaaccaccacgatcgttcccacctcgacccccgctggtcctggcttccgttccagcttcctcaacaagagtctgcgcaaacagattgccgaccaggaagctctcgaaagcggccaagactctgactctgactcggacgacaacgaaaacgacacaggtcttgtcgccggcgccacctttgctgctatgtacaaaaaggcagccgccaacacgcaagccaatactcgcgcaaatgctgtaccagcgtcaaacagcaccactagctccaagaagcgcaagagtgacgaggttcttccagctgcgattgaggccagcttgagcgctgccgctccaccgcacaaggtgtccaagctgggtgccgcgatgatggcggccaagacacccacgtccgctcaaccatctactgccaagaaagcaccagctgccggccctgcgtccaaactggagcaattccgcaacaacctcgctcatgttgctcgcagcaccggaagcggtgcgagtgccagctcggcttctcagtcagcgtcgtccgtcacgtcgcccaccatggaacaagcaccatccgcgccgctgcccgtcgtcaagccttcggatgcggccgtcgtgccattcgctcaagaagcgtcggcactgccttccattgaaagcttgaatgcttcgcattcgaaatcctcggattctggctccgactcgacggaaaaacttacggcaggtaccaagagtggcgagtcaaccatcaaggcttctgccatcccacgatcgcccactcgctctcctcgcgccgccgccacttcaccgatgcggcctccgcctcgcaccggcagtgtgcgtaaatctccacgcaaagtggccgagcagatcaacacaagcggacaagagtcggccagatcggcttcgccggggcccaaggtgaaagaaagtatagctcttttccagtcgagcagaaacggcggtctgactgataagcacacacgtagccctaccacgcagccccgaagtctcgcagcactctttggcagtcccaagcaagcgaccaaactaccgacgctgtcggccactgccggcagcagcaccaccccggctcactctccacccaagaactggcaaggaagtcgtttaccgtttgcacaaaccttccagttgcaacagcaagcgctgcgtgaggtggacaagtctttgccgcagacaccggccgaggagacggccgacagccagcgtgtcgtctcggcagcctcgacggtcttgtccacgtccaaggctgaaacagcggagagccaattcttcgacgcccgctcgagggagaacagtgccgagccagccgatgcgccagctccagcgccattggcggcgatgaaaggcgcgacggcacctgggaccagtgcacctcaagtggtcgccgaaaaggagtgcgtcatgcttgctgaggcacccatcgttgccaacaaggcatccagcattgtgaagactagctcgctttcaccaaagaaagtggcggccaaggcgcttgcgaaaactggaccgacctcgccgaccaaggcgagcagtctccgagctgcagccaacgccacgtcgccttccaagatcaaggcgtcggcgcgcacagcacagcagcatggcggaccgggactgggaccgggaccggcaccgtctggatcgccttcgagggcttggggcattggtgaaaagatcaaagggttcctcggattgcatgcctcgacgacgatccacccatctacagcagttgctcagaccaaggtatcggcgtctggcacattccagccgcgtgccaacgggtttgccacaaacacggctcatgcgagcgcttccgaacacaagactgcggctacagctacaccggcaccttgcattcctggcgctctgcttgctagccctccgccaacggccaagtctgcgcaggtcaactcaacaccctcaagcggtgctgcttcggcgacggccaacggaaagctcaccagcattgcgcgcgccgaaatgctgcgcaagaagcaggcagaggaggaggagcgcaaggctaaggacaagcaagagcgtcgaagaatgctgatggccaagaaggaagagcgtgccaaggcggtcgcggaggaggaaatcgaaaaggagaaccgcaaacgagcgcgtgagcagaacgaccgcctcggccgttccggcatcgaagacgccagcatcgcttcgtctaacggtgcaccggctgcgattcctgctgcgctcctgggcaatattgtcgtcggtggcggtggctccgcaatttctggcagtgttcacagcggctcagctagctcaaggacaagcacagtcagcaccgttagcagcggaccgcccacgcgaccatcaagtgctctcaatggaagctatgctggtgtcaagagtcgctacatgcagggagcggtccagcagcagcagcagcagccgccgccgcagcaacaaggcgaggaaagtagtaagaagcgtcgcgtcacaaatgacaaggaaggtatagcggcaggcttgcaagtgccattgaagcagcagccggcgcctcagcaatcacaaggtgcacctagcgctcgtcctggtgcttcggtggcgacgtcgcagatcggtgcacccaagatggttcgcacagtcagccagtcgtcgatacgatctggtgctgcctcacaagctgctgcccagcaacagcaacagcaacagcgcactcagccgccgccgcagatcaagctgggcatgaagccgagtgcggcgacgacagcagcagcaggagcaggatttggagcagcgccacgcataggcggagctggtgctacagccggcaacggcgcggctactgcgtcaatgatggcgaatgctggcaaagctgtttcgtcggcgcaacccgcggcagccaaggctatggcgaatgccaacttttgctcttcgaatccgttccagcaagccaagcagcagcaactgcagctgcagttgcaaaagcagaagcagcttcaaatgcagcaacagcagcaacagcaacagcaagttgcatccaacgcgatgcaggcgcaggcggctgctagtgcggcagcggtgcgagaattagagttgcatcgtgcggcgcaattgcaaatgcagatgcagaatgcagctggtggacggtatcaagagatggacgacgatgttgcaggcgatggcgacgaggtgctgccggagattgcatcggaatactcggactcggaagacgaagagacaattcagaagcgagcggcgatgccgttgtggacgcagggcgatatgctggatgcggcgctgttggcgcaatccacggtggatgccgatgagatctttggtattccacatggtcctgttgagctagaaaaaatcttgcctggcgagcgcacagcgaaccggatccgacgagcgcgaacttcgtcggccaactggtccgggcccgacggcctcgcgcagtgggagatcgacagatacaacaaacgcatgggcatcaagagtgcaggtgtccagttacatcgcgacccatctcagccgtctcggccagccgctatgccatccctctcgatccaccgacactcgatctcgcacaacccactcgctcccacctcgtccacctcgtccactgctccacacacagccatcaacgacgacgctcgtacaaatgcttag

SEQ ID NO: 14 - protein, 2147 aa, Artificial Sequence
  Aurorakinaseaktivator mit MBP-TAG
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISELSTSLYKKAGSAAAVLEENLYFQGSFTMLRVRFGACPLAFILTIITLRITPPPVGSSRFQLMHFVLVLAHLASRRLDSRPEKRCLHLHPPQIGSRHPSWYPRSYSDSDKLRPTSSADSSVSFLGSKSITLSSHADSSNRFSHRVPALDSSASTSAASSDSSSPLTPEERLLSHVNNMRRCCDMSLFHKNLQEQGIDFIADSLLHANDIFAKYATTSRTVASPSKLAGELLKSPTRPRTRARDAPAASGYPRGAFKVSIDPFVDAADVFDKENAPLQTRSSRSRTASTVEPQKGKGKARAKAIHAVEREASSPLSRPTRRVLGDVNTQKNETKQPTPQPKPTSKEATLKNIATDDDSMVIDSSLQAHLSPDDTMPRASTPPPPQASPPRILALHKPSKATQSAASLSGAISITDSDEDGSDGDSDADLHEEETGGLNDTVQHEVAEPTVTGNVVSRSSRSDDNTASFQPDINDTMPANDNTVSGTLIDPKLESARQAASVLLTSKIQQQKDANAMKPVKRTLASYGAKKSLPAAANNSGPSNVTTTIVPTSTPAGPGFRSSFLNKSLRKQIADQEALESGQDSDSDSDDNENDTGLVAGATFAAMYKKAAANTQANTRANAVPASNSTTSSKKRKSDEVLPAAIEASLSAAAPPHKVSKLGAAMMAAKTPTSAQPSTAKKAPAAGPASKLEQFRNNLAHVARSTGSGASASSASQSASSVTSPTMEQAPSAPLPVVKPSDAAVVPFAQEASALPSIESLNASHSKSSDSGSDSTEKLTAGTKSGESTIKASAIPRSPTRSPRAAATSPMRPPPRTGSVRKSPRKVAEQINTSGQESARSASPGPKVKESIALFQSSRNGGLTDKHTRSPTTQPRSLAALFGSPKQATKLPTLSATAGSSTTPAHSPPKNWQGSRLPFAQTFQLQQQALREVDKSLPQTPAEETADSQRVVSAASTVLSTSKAETAESQFFDARSRENSAEPADAPAPAPLAAMKGATAPGTSAPQVVAEKECVMLAEAPIVANKASSIVKTSSLSPKKVAAKALAKTGPTSPTKASSLRAAANATSPSKIKASARTAQQHGGPGLGPGPAPSGSPSRAWGIGEKIKGFLGLHASTTIHPSTAVAQTKVSASGTFQPRANGFATNTAHASASEHKTAATATPAPCIPGALLASPPPTAKSAQVNSTPSSGAASATANGKLTSIARAEMLRKKQAEEEERKAKDKQERRRMLMAKKEERAKAVAEEEIEKENRKRAREQNDRLGRSGIEDASIASSNGAPAAIPAALLGNIVVGGGGSAISGSVHSGSASSRTSTVSTVSSGPPTRPSSALNGSYAGVKSRYMQGAVQQQQQQPPPQQQGEESSKKRRVTNDKEGIAAGLQVPLKQQPAPQQSQGAPSARPGASVATSQIGAPKMVRTVSQSSIRSGAASQAAAQQQQQQQRTQPPPQIKLGMKPSAATTAAAGAGFGAAPRIGGAGATAGNGAATASMMANAGKAVSSAQPAAAKAMANANFCSSNPFQQAKQQQLQLQLQKQKQLQMQQQQQQQQQVASNAMQAQAAASAAAVRELELHRAAQLQMQMQNAAGGRYQEMDDDVAGDGDEVLPEIASEYSDSEDEETIQKRAAMPLWTQGDMLDAALLAQSTVDADEIFGIPHGPVELEKILPGERTANRIRRARTSSANWSGPDGLAQWEIDRYNKRMGIKSAGVQLHRDPSQPSRPAAMPSLSIHRHSISHNPLAPTSSTSSTAPHTAINDDARTNA

SEQ ID NO: 15 - DNA, 1707 bp, Artificial Sequence
  Aurorakinaseaktivator (C-Terminus) mit MBP-TAG
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattatcaacaagtttgtacaaaaaagcaggctccgcggccgccgtactcgaggaaaacctgtattttcagggctccttcacctactcggactcggaagacgaagagacaattcagaagcgagcggcgatgccgttgtggacgcagggcgatatgctggatgcggcgctgttggcgcaatccacggtggatgccgatgagatctttggtattccacatggtcctgttgagctagaaaaaatcttgcctggcgagcgcacagcgaaccggatccgacgagcgcgaacttcgtcggccaactggtccgggcccgacggcctcgcgcagtgggagatcgacagatacaacaaacgcatgggcatcaagagtgcaggtgtccagttacatcgcgacccatctcagccgtctcggccagccgctatgccatccctctcgatccaccgacactcgatctcgcacaacccactcgctcccacctcgtccacctcgtccactgctccacacacagccatcaacgacgacgctcgtacaaatgcttag

SEQ ID NO: 16 - protein, 568 aa, Artificial Sequence
  Aurorakinaseaktivator (C-Terminus) mit MBP-TAG
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISELSTSLYKKAGSAAAVLEENLYFQGSFTYSDSEDEETIQKRAAMPLWTQGDMLDAALLAQSTVDADEIFGIPHGPVELEKILPGERTANRIRRARTSSANWSGPDGLAQWEIDRYNKRMGIKSAGVQLHRDPSQPSRPAAMPSLSIHRHSISHNPLAPTSSTSSTAPHTAINDDARTNA